(19) United States
(12) Patent Application Publication
(10) Pub. No.: US 2005/0260730 A1
(43) Pub. Date: Nov. 24, 2005

(54) CDK2/cyclin A crystals and uses thereof

(76) Inventor: Peter Martin Fischer, Angus (GB)

Correspondence Address: LAHIVE & COCKFIELD, LLP., 28 STATE STREET, BOSTON, MA, 02109, US

(21) Appl. No.: 10/914,842

(22) Filed: Aug. 10, 2004

(30) Foreign Application Priority Data
Oct. 20, 2003 (GB) 0324465.4

(51) Int. Cl. G06F 19/00; G01N 33/48; G01N 33/50; C12N 9/12

(52) U.S. Cl. 435/194; 702/19

(57) ABSTRACT

The present invention relates to a method of forming a crystal complex of CDK2/cyclin A and a cyclin binding groove peptide oligomer using a technique involving ligand exchange within the protein crystal. The invention further relates to novel crystal complexes of CDK2/cyclin A with various peptide oligomers, methods of identifying cyclin binding groove ligands, and methods of treating proliferative disorders.

### RELATED APPLICATION

This application claims priority to application number 0324465.4 filed Oct. 20, 2003 filed in the United Kingdom. The contents of the aforementioned application is hereby incorporated by reference.

The present invention relates to a method of forming a crystal. More specifically, the invention relates to a method of forming a crystal of CDK2/cyclin A and a cyclin binding groove ligand. The invention further relates to novel crystal complexes of CDK2/cyclin A with various peptide oligomers.

### BACKGROUND TO THE INVENTION

Human cancer cells are characterised by the loss of cell cycle checkpoint regulation, leading to cell proliferation under conditions where non-transformed cells cannot enter and pass through the cell cycle. CDKs and their natural inhibitors, the CDK tumour suppressor proteins (CDKIs), are central to cell cycle regulation and their functions are commonly altered in tumour cells (Ortega et al., 2002). Deregulation of CDK2 and CDK4 through inactivation of CDKIs such as p16^{INK4a}, p21^{WAF1}, p27^{KIP1}, and p57^{KIP2}, thus provides a means for cancer cells to override the G1 checkpoint. Reinstatement of CDK inhibition therefore represents an opportunity for pharmacological interference with tumour progression. This is particularly attractive as one consequence of unchecked CDK2 activity is persistent E2F transcriptional activity, which, unless terminated in a timely fashion during S-phase, constitutes a powerful apoptotic signal (Lees and Weinberg, 1999). Because E2F deactivation occurs through phosphorylation by CDK2/CA, tumour cells will be selectively sensitive to inhibition. One method of CDK2/ CA inhibition is through the use of peptides that block binding of both pRb and E2F to the CDK2/CA complex (Fischer and Lane, 2000).

Recognition of both of these substrates, as well as CDKIs, by the CDK2/CA complex depends on a shallow hydrophobic surface binding site in the cyclin A subunit, termed the cyclin groove (Schulman et al., 1998). Recognition of this groove is mediated through certain sequences, designated the CBM (Chen et al., 1996). This motif represents a consensus of the cyclin groove binding sequences found in a variety of CDK2/CA partner proteins, including the CDKIs, pRb proteins, E2F transcription factors, p53, HDM2, the CDK-activating kinase CDK7, etc. This sequence is commonly referred to as the RXL or ZRXL (SEQ. ID No: 7) motif, where Z and X denote basic residues (Fischer et al., 2003). Following a detailed study with peptides representing various sequences from cyclin A binding proteins, the definition of the motif has recently been refined (McInnes et al., 2003): ZRXLYY′ (SEQ. ID No: 8), where Y and Y′ are hydrophobic residues. Further studies have concluded that the cyclin binding motif from p21 is more accurately described as a cluster of hydrophobic residues covering the LYY′ portion of the sequence above (Wohlschlegel et al, 2001). However, other positions in the motif are seen and differences are observed in the relative importance of particular residues, most probably due to differences in experimental design.

When CBM peptides are conjugated to cellular delivery vectors (Fischer et al., 2001), the resulting peptide constructs cause cell cycle arrest and selective apoptosis in tumour cells in vitro (Ball et al., 1996; Bonfanti et al., 1997; Chen et al., 1999) and, as shown more recently, in vivo (Mendoza et al., 2003). Inhibition of CDK2 activity through blocking of substrate recruitment is also likely to provide kinase selectivity, which is more difficult to achieve with traditional ATP-competitive molecules (Fischer, 2001), since the cyclin-dependent substrate recognition, and hence phosphorylation, appears to be unique to CDKs 2 and 4.

A number of CDK2/CA complex X-ray crystal structures with CBM peptides have been reported. The first of these included a 68-residue p27^{KIP1} fragment containing the ³⁰RNLFG³⁴ (SEQ. ID No: 9) sequence (Russo et al., 1996). A later study used a peptide with the sequence ⁶⁵⁸RRLFGEDPPKE⁶⁶⁸ (SEQ. ID No: 10) from p107, a pRb-family protein (Brown et al., 1999). The most recent complexes reported included the peptides ³⁷⁶STSRHKKLMFK³⁸⁶ (SEQ. ID No: 11) (p53), ⁸⁶⁸PPKPLKKLRFD⁸⁷⁸ (SEQ. ID No: 12) (pRb), ²⁵KPSACRNLFGP³⁵ (SEQ. ID No: 13) (p27^{KIP1}), ⁸⁷PVKRRLDLE⁹⁵ (SEQ. ID No: 14) (E2F), and ⁶⁵³AGSAKRRLFGE⁶⁶³ (SEQ. ID No: 15) (p107) (Lowe et al., 2002). However, to date there have been no reports of crystallographic complexes of CDK2/CA with various peptides having the p21^{WAF1} motif ¹⁵⁵RRLIF¹⁵⁹ (SEQ. ID No: 16).

A comparison of a series of octapeptides derived from the p21^{WAF1} CBM-containing sequences, as well as those found in p107, E2F1, and p27^{KIP1}, showed (Zheleva et al., 2002) that the p21^{WAF1 55}RRLIF¹⁵⁹ (SEQ. ID No: 16) motif was the most effective in cyclin A inhibitor peptides. In this motif the Arg¹⁵⁵ residue was demonstrated to contribute extensively to binding. The optimised bioactive peptide H-His-Ala-Lys-Arg-Arg-Leu-Ile-Phe-NH₂, (SEQ. ID No: 17) a low nanomolar CDK2/CA inhibitor (Zheleva et al., 2002), corresponds to the p21^{WAF1}(152-159) sequence, with a Ser¹⁵³-Ala mutation, which was found to enhance potency significantly. These observations rendered this sequence a good candidate for structural studies, as it provides the best template for the design of peptidomimetics.

The present invention relates to an approach for developing potent, smaller, and less peptidic inhibitors of CDK2/CA. In particular, the invention seeks to provide an improved method of preparing crystals complexes of CDK2/CA with p21^{WAF}-derived peptide analogues. The invention further seeks to use X-ray crystallography in conjunction with in vitro activity results to provide a comprehensive description of the requirements of each position in the CBM and to define a series of design principles for the structure-guided design of small molecule cyclin groove inhibitors.

### SUMMARY OF THE INVENTION

A first aspect of the invention provides a method of preparing a crystal comprising CDK2/cyclin A and a ligand, L, said method comprising the steps of:
(i) co-crystallising CDK2/cyclin A and a first ligand, L′, to form a crystal;
(ii) removing at least a portion of said first ligand, L′ from said crystal;
(iii) contacting said crystal with a second ligand, L, to form a crystal comprising CDK2/cyclin A and ligand, L.

Further aspects of the invention relate to a crystal obtainable by the above method, and the use thereof in the preparation of a medicament for treating cancer, leukemia, a viral disorder, stroke, glomerulonephritis, alopecia, diabetes, a CNS disorder, rheumatoid arthritis, psoriasis or chronic obstructive pulmonary disorder.

Another aspect of the invention relates to the use of a crystal obtainable by the above method in an assay for identifying further ligands capable of binding to the cyclin binding groove of CDK2/cyclin A.

Yet another aspect of the invention relates to a crystal comprising CDK2/cyclin A and a cyclin binding groove peptide selected from the following:

(SEQ ID NO. 1)

H-Ala-Ala-Abu-Arg-Ser-Leu-Ile-(p-F-Phe)-NH₂;

(SEQ ID NO. 2)

H-Arg-Arg-Leu-Ile-Phe-NH₂;

(SEQ ID NO. 3)

Ac-Arg-Arg-Leu-Asn-(m-Cl-Phe)-NH₂;

(SEQ ID NO. 4)

H-Arg-Arg-Leu-Asn-(p-F-Phe)-NH₂;

(SEQ ID NO. 5)

H-Cit-Cit-Leu-Ile-(p-F-Phe)-NH₂;
and

(SEQ ID NO. 6)

H-Arg-Arg-Leu-Ile-(p-F-Phe)-NH₂.

Another aspect relates to a method of screening for a ligand capable of binding to the cyclin binding groove of CDK2/cyclin A, wherein said method comprises the use of a crystal according to the invention, or obtainable by the method according to the invention. The invention also relates to a ligand identified by such a method.

A further aspect relates to the use of a ligand identifed by said method in the preparation of a medicament for treating cancer, leukemia, a viral disorder, stroke, glomerulonephritis, alopecia, diabetes, a CNS disorder, rheumatoid arthritis, psoriasis or chronic obstructive pulmonary disorder.

Another aspect relates to the use of a crystal according to the invention in the preparation of a medicament for treating cancer, leukemia, a viral disorder, stroke, glomerulonephritis, alopecia, diabetes, a CNS disorder, rheumatoid arthritis, psoriasis or chronic obstructive pulmonary disorder.

### BRIEF DESCRIPTION OF THE FIGURES

FIG. 1 shows a ribbon representation of the non-crystallographic dimer of the ternary CDK2/CA/peptide 2 complex. In more detail, FIG. 1 shows CDK2 molecules (black), cyclin A subunits (grey), bound peptides (stick representation) and ATP binding sites.

FIG. 2 shows replacement of one peptide with another in the CBG of a CDK2/CA crystal. 1Fo-1Fc difference electron density maps after 2 days soaking in mother liquor.

FIG. 3 shows the electron density maps of peptides 1-5. The 2Fo-1Fc maps are contoured at a level corresponding to 1.2 σ for the peptides bound to the non-occluded cyclin groove. (a) Superimposition of the structures of peptide 2 in the free and partially occluded CBG shows the differences of the two copies of the peptide. (b) Overlay of the bound conformations of peptides 1-5.

FIG. 4 shows (a) H-bonds involving the backbone of peptide 2 (b) Effect of P¹ side chains: A charged P¹ side chain affects the position and flexibility of the Leu³ side chain through van der Waal contacts, effectively locking the Leu³ side chain between side chains P¹ and P⁵. Mutation at P¹ from Arg (b1) to Cit (b2) results in a more flexible side chain at P¹, leading to a removal of the Leu³ lock, whose side chain is now more flexible (refer FIG. 3, electron density map 5). (c) Contacts of the Leu³ side chain in the peptide 2 structure. (d) Superimposition of the cyclin A-bound H-Arg-Leu-Ile-Phe-NH₂ (SEQ, ID No: 2) (CPK colouring peptide and the corresponding p27^{KIP1} peptide segment ³⁰Arg-Asn-Leu-Phe-Gly³⁴ (SEQ. ID No: 9 ) (PDB accession code 1JSU). (e) Van der Waals surfaces of the Ac-Arg-Arg-Asn-(m-Cl-Phe)-NH₂ (SEQ. ID No: 4) and peptide 4 complex structures surrounding the Phe⁵ site. The fluorine and chlorine surfaces match exactly at the interface with the cyclin A surface.

### DETAILED DESCRIPTION

As mentioned above, in one aspect, the invention relates to method of preparing a crystal comprising CDK2/cyclin A and a ligand, L.

Advantageously, the co-crystallisation of protein with a ligand, followed by “ligand exchange” provides a way of studying binding sites involved in protein-protein interactions that would otherwise be difficult to elucidate using conventional techniques such as “soaking” (for example, where native protein crystals are not readily obtainable, or where protein-recognition sites are occupied by neighbouring molecules in the crystal lattice), or “co-crystallisation”.

In particular, the present invention provides a general method of preparing crystals of CDK2/CA and a ligand, L, for complexes that are particularly difficult to prepare (or are indeed unobtainable) by standard techniques known in the art.

As used herein, the term “crystal” means a structure (such as a three dimensional (3D) solid aggregate) in which the plane faces intersect at definite angles and in which there is a regular structure (such as internal structure) of the constituent chemical species. Thus, the term “crystal” can include any one of: a solid physical crystal form such as an experimentally prepared crystal, a 3D model based on the crystal structure, a representation thereof such as a schematic representation thereof or a diagrammatic representation thereof, or a data set thereof for a computer.

In one aspect, the crystal is usable in X-ray crystallography techniques. Here, the crystals used can withstand exposure to X-ray beams used to produce a diffraction pattern data necessary to solve the X-ray crystallographic structure.

In one preferred embodiment, step (ii) comprises eluting the crystal obtained in step (i) with a solvent.

Preferably, step (iii) comprises soaking the crystal obtained in step (ii) with a solution of ligand, L.

In one preferred embodiment, steps (ii) and (iii) are carried out sequentially.

In an alternative preferred embodiment, steps (ii) and (iii) are carried out simultaneously.

Preferably, step (ii) comprises eluting said first crystal with a solvent to remove substantially all of said first ligand, L′.

The presently described method of ligand exchange is effective in the case where L has a stronger affinity for the cyclin binding groove than L′. Surprisingly, the presently described method is also effective in the case where L has a weaker affinity than L′. In other words, using the present method it is possible to replace a higher-affinity ligand with a lower-affinity ligand.

In a preferred embodiment, said first ligand, L′, and said second ligand, L, are different and are each capable of binding to the cyclin binding groove of CDK2/cyclin A.

The ligands of the present invention may be natural or synthetic. The term “ligand” also refers to a chemically modified ligand.

As used herein, the term “ligand” includes, but is not limited to, a compound which may be obtainable from or produced by any suitable source, whether natural or not. The ligand may be designed or obtained from a library of compounds which may comprise peptides, as well as other compounds, such as small organic molecules and particularly new lead compounds. By way of example, the ligand may be a natural substance, a biological macromolecule, or an extract made from biological materials such as bacteria, fungi, or animal (particularly mammalian) cells or tissues, an organic or an inorganic molecule, a synthetic ligand a semi-synthetic ligand, a structural or functional mimetic, a peptide, a peptidomimetic, a derivatised ligand, a peptide cleaved from a whole protein, or a peptides synthesised synthetically (such as, by way of example, either using a peptide synthesizer or by recombinant techniques or combinations thereof), a recombinant ligand, a natural or a non-natural ligand a fusion protein or equivalent thereof and mutants, derivatives or combinations thereof.

The ligand may also comprise “modified peptides”, which are peptides derived from unnatural amino acids. The term “unnatural amino acid” refers to a derivative of an amino acid and may for example include alpha and alpha-disubstituted amino acids, N-alkyl amino acids, lactic acid, halide derivatives of natural amino acids such as trifluorotyrosine, p-Cl-phenylalanine, p-Br-phenylalanine, p-I-phenylalanine, L-allyl-glycine, β-alanine, L-α-amino butyric acid, L-γ-amino butyric acid, L-α-amino isobutyric acid, L-ε-amino caproic acid, 7-amino heptanoic acid, L-methionine sulfone, L-norleucine, L-norvaline, p-nitro-L-phenylalanine, L-hydroxyproline, L-thioproline, methyl derivatives of phenylalanine (Phe) such as 4-methyl-Phe, pentamethyl-Phe, L-Phe (4-amino), L-Tyr (methyl), L-Phe (4-isopropyl), L-Tic (1,2,3,4-tetrahydroisoquinoline-3-carboxyl acid), L-diaminopropionic acid and L-Phe (4-benzyl).

The term “derivative” or “derivatised” as used herein includes the chemical modification of a ligand. Typical chemical modifications may include, for example, the replacement of a hydrogen by a halo group, an alkyl group, an acyl group or an amino group.

Once a ligand capable of interacting with a key amino acid residue in the cyclin binding groove has been identified, further steps may be carried out either to select and/or to modify compounds and/or to modify existing compounds, to modulate the interaction with the key amino acid residues.

By way of example, once a ligand has been optimally selected or designed, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit to the cyclin binding groove by the same computer methods described above.

Preferably, positions for substitution are selected based on the predicted binding orientation of a test compound to the cyclin binding groove of CDK2/cyclin A. The ligand of the present invention or mimetics thereof may be produced using chemical methods to synthesize the ligand in whole or in part. For example, peptides can be synthesized by solid phase techniques, cleaved from the resin, and purified by preparative high performance liquid chromatography (e.g., Creighton (1983) Proteins Structures And Molecular Principles, WH Freeman and Co, New York N.Y.). The composition of the synthetic peptides may be confirmed by amino acid analysis or sequencing (e.g., the Edman degradation procedure; Creighton, supra).

Direct synthesis of the ligand or mimetics thereof can be performed using various solid-phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Additionally, the amino acid sequences obtainable from the ligand, or any part thereof, may be altered during direct synthesis and/or combined using chemical methods with a sequence from other subunits, or any part thereof, to produce a variant ligand.

In an alternative embodiment of the invention, the coding sequence of the ligand or mimetics thereof may be synthesized, in whole or in part, using chemical methods well known in the art (see Caruthers M H et al (1980) Nuc Acids Res Symp Ser 215-23, Horn T et al (1980) Nuc Acids Res Symp Ser 225-232).

Hence, the ligands may be chemically synthesised or they may be prepared using recombinant techniques.

In one preferred aspect, the ligand is prepared by the use of chemical synthesis techniques.

In another preferred aspect, the ligands of the present invention may be produced from host cells using recombinant techniques.

A wide variety of host cells can be employed for expression of the nucleotide sequences encoding the ligands of the present invention. These cells may be both prokaryotic and eukaryotic host cells. Suitable host cells include bacteria such as E. coli, yeast, filamentous fungi, insect cells, mammalian cells, typically immortalized, e.g., mouse, CHO, human and monkey cell lines and derivatives thereof. Preferred host cells are able to process the expression products to produce an appropriate mature polypeptide. Processing includes but is not limited to glycosylation, ubiquitination, disulfide bond formation and general post-translational modification.

In one embodiment of the present invention, the ligand may be a chemically modified ligand.

The chemical modification of a ligand and/or a key amino acid residue of the present invention may either enhance or reduce hydrogen bonding interaction, charge interaction, hydrophobic interaction, Van Der Waals interaction or dipole interaction between the ligand and the key amino acid residue(s) of the cyclin binding groove.

More preferably, said first ligand, L′, and said second ligand, L, are each cyclin binding groove inhibitors.

Preferably, the cyclin binding groove is defined by the structural coordinates of the following amino acid residues of human CDK2/cyclin A: Met²¹⁰, Ile²¹³, Leu²¹⁴, Trp²¹⁷, Leu²⁵³, Glu²²⁰, Val²²¹, Ile²⁸¹ and Gln²⁵⁴, or a homologue thereof.

As used herein, the term “structural co-ordinates” refer to a set of values that define the position of one or more amino acid residues with reference to a system of axes.

As used herein, the term “homologue” refers to the ligand binding domain of the cyclin binding groove, or a portion thereof, which may have deletions, insertions or substitutions of amino acid residues as long as the binding specificity of the molecule is retained. In this regard, deliberate amino acid substitutions may be made on the basis of similarity in polarity, charge, solubility, hydrophobicity, hydrophilicity, and/or the amphipathic nature of the residues as long as the binding specificity of the ligand binding domain is retained.

In one particularly preferred embodiment, said first ligand, L′, and said second ligand, L, are each peptides.

More preferably, the peptides are p21^{WAF}-derived peptide analogues.

Previous studies by the applicant delineated the components of the p21^{WAF1} CBM (Zheleva et al., 2002) and arrived at a complete description of the motif through both extensive residue replacements, as well as by molecular modelling (McInnes et al., 2003). It was proposed that residues C-terminal to the ZRXL (SEQ. ID NO: 7) motif are also critical for cyclin A binding and hence the motif would be better described by the sequence ZRXLYY′ (SEQ. ID NO: 8), where either Y and/or Y′ are hydrophobic residues. The studies described herein confirm this crystallographically and in addition, reveal that the ZRXLYY′ (SEQ. ID NO: 8) motif can be further truncated to RXLYY′ (SEQ. ID NO: 18), since the pentapeptide H-Arg-Arg-Leu-Ile-Phe-NH₂ (SEQ. ID NO: 2) retains considerable potency. In the present study, several CBM peptides, including those identified previously (McInnes et al., 2003), containing unnatural amino acids and which resulted in potency enhancements, were crystallised with CDK2/CA, and their interactions with the cyclin groove described. The structural interpretation of the potency differences between various CBM peptides illustrates the determinants of cyclin groove binding, and shows that small substitutions in the peptide structures can result in profound differences in activity and in the bound conformation.

The peptide residue numbering system used throughout this work is based on the minimally active pentapeptide sequence, where amino acid residues are denoted Arg¹-Arg²-Leu³-Ile⁴-Phe⁵ (SEQ. ID NO: 16) and residues extending from the N- and C-termini are numbered −1 and 6, respectively. The cyclin groove components involved in binding specific residues are denoted similarly, e.g. site 1 for elements interacting with the residue at position (P¹) of the peptide ligand.

In one especially preferred embodiment, said first ligand, L′, and said second ligand, L, are selected from:

(SEQ ID NO. 1; “Peptide 1”)

H-Ala-Ala-Abu-Arg-Ser-Leu-Ile-(p-F-Phe)-NH₂;

(SEQ ID NO. 2; “Peptide 2”)

H-Arg-Arg-Leu-IIe-Phe-NH₂;

(SEQ ID NO. 3; “Peptide 3”)

Ac-Arg-Arg-Leu-Asn-(m-Cl-Phe)-NH₂;

(SEQ ID NO. 4; “Peptide 4”)

H-Arg-Arg-Leu-Asn-(p-F-Phe)-NH₂;
and

(SEQ ID NO. 5; “Peptide 5”)

H-Cit-Cit-Leu-Ile-(p-F-Phe)-NH₂;

wherein L and L′ are different.

In one particularly preferred embodiment, said first ligand, L′, is selected from:

| | |
|---|---|
| H-Arg-Arg-Leu-Ile-Phe-NH₂; | (SEQ ID NO: 2) |
| and | |
| H-Arg-Arg-Leu-Asn-(p-F-Phe)-NH₂. | (SEQ ID NO. 4) |

In another particularly preferred embodiment, said second ligand, L, is selected from:

(SEQ ID NO. 1)

H-Ala-Ala-Abu-Arg-Ser-Leu-Ile-(p-F-Phe)-NH₂;

(SEQ ID NO. 3)

Ac-Arg-Arg-Leu-Asn-(m-Cl-Phe)-NH₂;
and

(SEQ ID NO. 5)

H-Cit-Cit-Leu-Ile-(p-F-Phe)-NH₂.

In one particularly preferred embodiment, said first ligand, L′, and said second ligand, L, are each pentamers.

Preferably, residues 1, 3 and 5 of ligand L are capable of interacting with the cyclin binding groove of CDK2/cyclin A.

More preferably, residue 1 of ligand L is capable of interacting with Glu²²⁰ of the cyclin binding groove of CDK2/cyclin A.

Even more preferably, residue 1 is Arg.

Preferably, residue 3 of ligand L is capable of interacting with Gln²⁵⁴, Trp²¹⁷ and Leu ²¹⁴ of the cyclin binding groove of CDK2/cyclin A.

More preferably, residue 3 is Leu.

In one preferred embodiment of the invention, the crystal is of space group P2₁2₁2 ₁.

In one particularly preferred embodiment, ligand L is H-Ala-Ala-Abu-Arg-Ser-Leu-Ile-(p-F-Phe)-NH₂ (SEQ ID NO. 1) and said crystal comprises a unit cell having unit dimensions a=74.5 Å, b=114.6 Å, c=157.0 Å.

In another particularly preferred embodiment, ligand L is H-Arg-Arg-Leu-Ile-Phe-NH₂ (SEQ ID NO. 2) and said crystal comprises a unit cell having unit dimensions a=74.2 Å, b=113.9 Å, c=155.3 Å.

In yet another particularly preferred embodiment, ligand L is Ac-Arg-Arg-Leu-Asn-(m-Cl-Phe)-NH₂ (SEQ ID NO. 3) and said crystal comprises a unit cell having unit dimensions a=74.5 Å, b=114.0 Å, c=156.2 Å.

In another particularly preferred embodiment, ligand L is H-Arg-Arg-Leu-Asn-(p-F-Phe)-NH₂ (SEQ ID NO. 4) and said crystal comprises a unit cell having unit dimensions a=73.5 Å, b=113.0 Å, c=153.1 Å.

In another particularly preferred embodiment, ligand L is H-Cit-Cit-Leu-Ile-(p-F-Phe)-NH₂ (SEQ ID NO. 5) and said crystal comprises a unit cell having unit dimensions a=74.5 Å, b=113.5 Å, c=154.5 Å.

Another aspect of the invention relates to a crystal obtainable by the above described method.

A further aspect of the invention relates to the use of a crystal obtainable by the above-described method in the preparation of a medicament for treating cancer, leukemia, a viral disorder, stroke, glomerulonephritis, alopecia, diabetes, a CNS disorder, rheumatoid arthritis, psoriasis or chronic obstructive pulmonary disorder.

As used herein the phrase “preparation of a medicament” includes the use of a crystal directly as the medicament in addition to its use in a screening programme for further therapeutic agents or in any stage of the manufacture of such a medicament.

Another aspect relates to the use of a crystal obtainable by the above-described method in an assay for identifying further ligands capable of binding to the cyclin binding groove of CDK2/cyclin A.

Yet another aspect of the invention relates to a crystal comprising CDK2/cyclin A and a cyclin binding groove peptide selected from the following:

(SEQ ID NO. 1)

H-Ala-Ala-Abu-Arg-Ser-Leu-Ile-(p-F-Phe)-NH₂;

(SEQ ID NO. 2)

H-Arg-Arg-Leu-Ile-Phe-NH₂;

(SEQ ID NO. 3)

Ac-Arg-Arg-Leu-Asn-(m-CJ-Phe)-NH₂;

(SEQ ID NO. 4)

H-Arg-Arg-Leu-Asn-(p-F-Phe)-NH₂;

(SEQ ID NO. 5)

H-Cit-Cit-Leu-Ile-(p-F-Phe)-NH₂;
and

(SEQ ID NO. 6)

H-Arg-Arg-Leu-Ile-(p-F-Phe)-NH₂.

Preferably, the crystal is of space group P2₁2₁2₁.

Preferably, the cyclin binding groove is defined by the structural coordinates of the following amino acid residues of CDK2/cyclin A: Met²¹⁰, Ile²¹³, Leu²¹⁴, Trp²¹⁷, Leu²⁵³, Glu²²⁰, Val²²¹, Ile²⁸¹ and Gln²⁵⁴, or a homologue thereof.

Preferably, residues 1, 3 and 5 of the cyclin binding groove peptide are capable of interacting with the cyclin binding groove of CDK2/cyclin A.

In one preferred embodiment, residue 1 of the cyclin binding groove peptide is capable of interacting with Glu²²⁰ of the cyclin binding groove of CDK2/cyclin A.

In another preferred embodiment, residue 3 of the cyclin binding groove peptide is capable of interacting with Gln²⁵⁴, Trp²¹⁷ and Leu²¹⁴ of the cyclin binding groove of CDK2/cyclin A.

In one particularly preferred embodiment, ligand L is H-Ala-Ala-Abu-Arg-Ser-Leu-Ile-(p-F-Phe)-NH₂ (SEQ ID NO. 1) and said crystal comprises a unit cell having unit dimensions a=74.5 Å, b=114.6 Å, c=157.0 Å.

In another particularly preferred embodiment, ligand L is H-Arg-Arg-Leu-Ile-Phe-NH₂ (SEQ ID NO. 2) and said crystal comprises a unit cell having unit dimensions a=74.2 Å, b=113.9 Å, c=155.3 Å.

In another particularly preferred embodiment, ligand L is Ac-Arg-Arg-Leu-Asn-(m-Cl-Phe)-NH₂ (SEQ ID NO. 3) and said crystal comprises a unit cell having unit dimensions a=74.5 Å, b=114.0 Å, c=156.2 Å.

In another particularly preferred embodiment, ligand L is H-Arg-Arg-Leu-Asn-(p-F-Phe)-NH₂ (SEQ ID NO. 4) and said crystal comprises a unit cell having unit dimensions a=73.5 Å, b=113. Å, c=153.1 Å.

In another particularly preferred embodiment, ligand L is H-Cit-Cit-Leu-Ile-p-F-Phe)-NH₂ (SEQ ID NO. 5) and said crystal comprises a unit cell having unit dimensions a=74.5 Å, b=113.5 Å, c=154.5 Å.

Yet another aspect relates to the use of a crystal according to the invention in the preparation of a medicament for treating cancer, leukemia, a viral disorder, stroke, glomerulonephritis, alopecia, diabetes, a CNS disorder, rheumatoid arthritis, psoriasis or chronic obstructive pulmonary disorder.

Crystal Soaking and Ligand Exchange

Despite considerable efforts, crystals of uncomplexed CDK2/cyclin A could not be obtained in the required crystal form. Initially, published crystallisation conditions (Jeffrey et al., 1995) were employed. However, attempts to introduce peptides by soaking of these crystals were not successful and resulted in crystal lattice disorder and X-ray diffraction at a resolution of 5 Å at best.

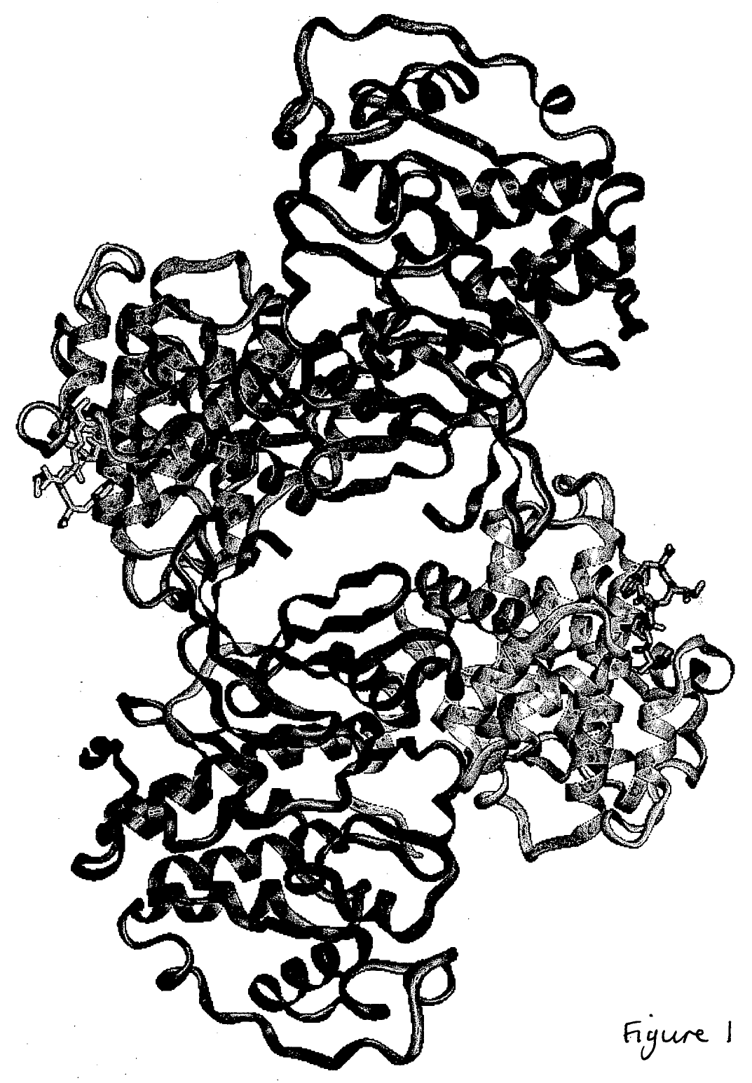

All peptide complex structures presented here were solved using a novel crystal form, which has space group P2₁2₁2₁ and contains two copies of the binary protein complex in the crystallographic asymmetric unit. Of the two peptide ligands in this complex, one (FIG. 1) is fully exposed to solvent channels in the crystal, while the second peptide site is partially occluded.

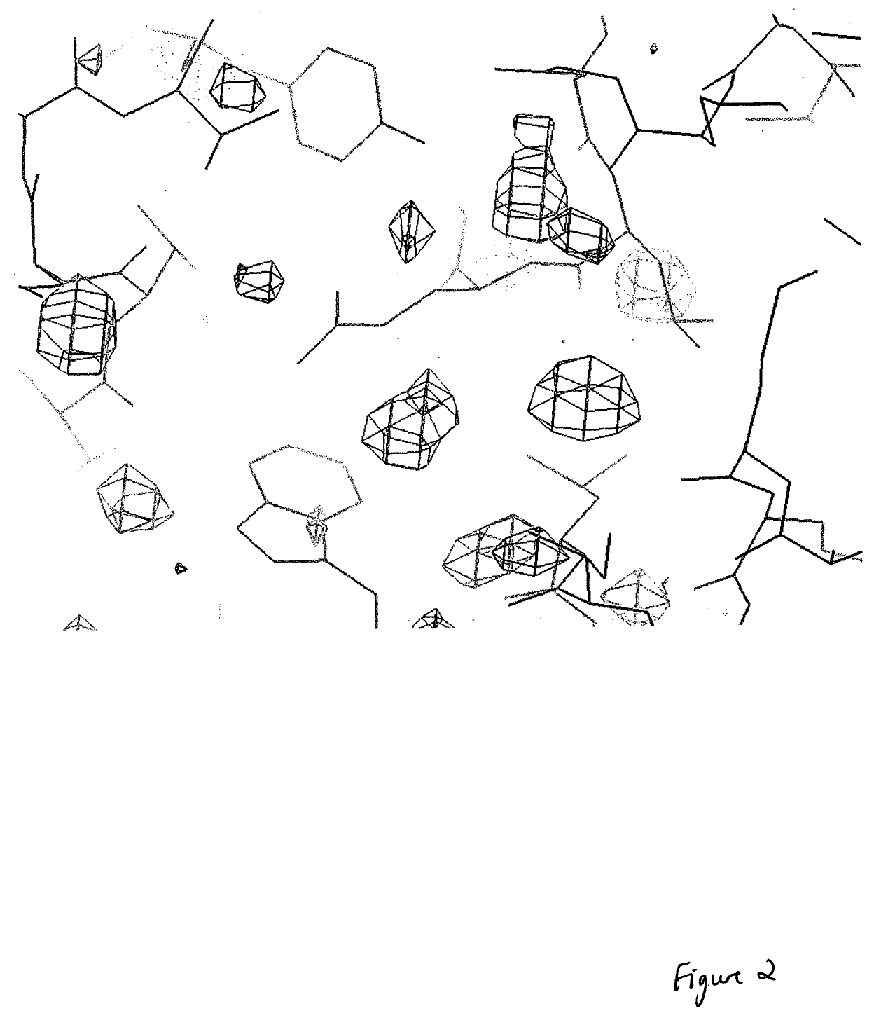

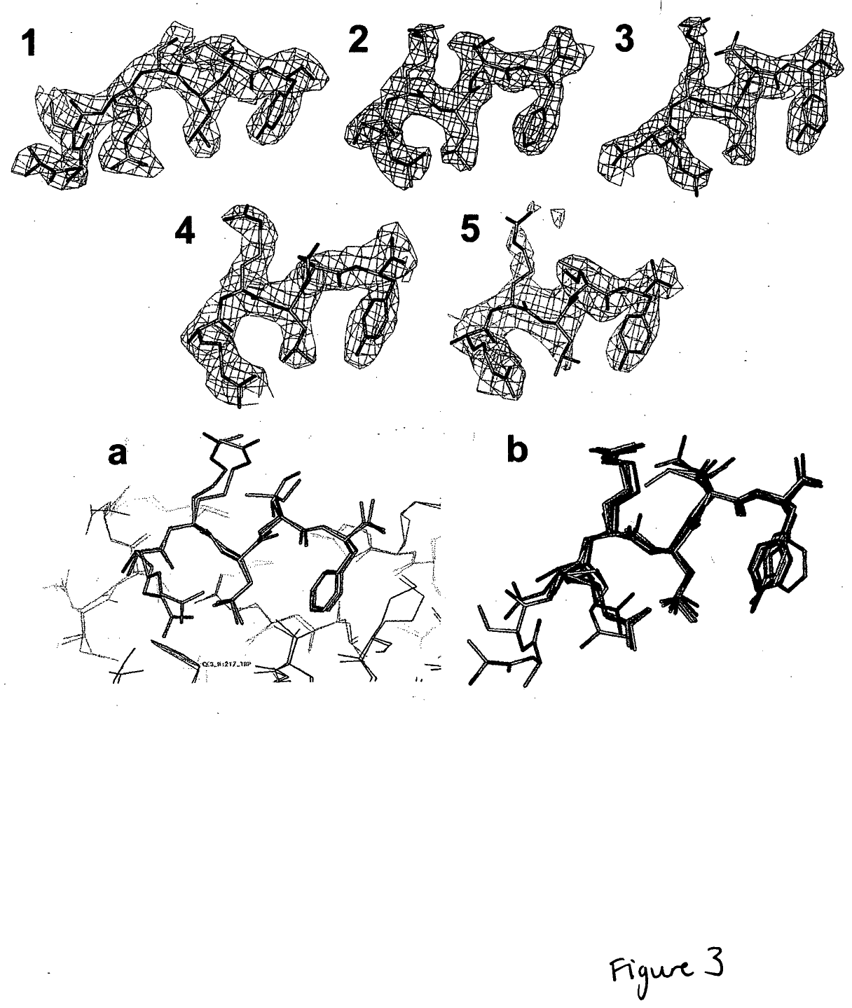

Peptides 2 and 4 were co-crystallised with CDK2/cyclin A, while the remaining peptide complexes were prepared using the so-called “ligand exchange” procedure. A crystal containing H-Arg-Arg-Leu-Ile-Phe-NH₂ (SEQ. ID NO: 2) was soaked extensively against precipitant solution to elute the original peptide. Examination of electron density difference maps around the cyclin binding groove regions showed that the original co-crystallised peptide (FIG. 3, peptide 2) had indeed been washed out (FIG. 2). A second crystal was treated in the same way and then soaked in approximately 10 μL mother liquor solution containing 1 mM of a different peptide. The structure obtained showed that this peptide had been able to diffuse into the crystal lattice and to dock in the cyclin groove (FIG. 3; peptide 1, 3 and 5). As the binding groove of one cyclin A molecule in the crystallographic unit is partially blocked by a neighbouring CDK molecule, diffusion of the peptide ligand to this site was incomplete, even after three days of soaking. For this reason, ligand occupancy levels at the partially occluded binding groove are lower than at the free site in some of our structures. Due to this differential occupancy of the two sites in the asymmetric unit, only the fully accessible cyclin groove was used to compare the different bound peptide ligands. Crystallographic data for several novel p21^{WAF1}-derived peptide structures are summarised in Table 1.

In general, the time required for ligand soaking experiments can vary between seconds and days. In systems where no protein rearrangement is necessary (e.g. dipeptide ligands soaked into crystals of cyclophilin), binding was found to take place within seconds (Wu et al., 2001). This contrasts with soaking times of days required for the binding of some CDK2 active site inhibitors, which require significant changes in the protein conformation before binding can occur (Wu et al., 2003).

Diffusion of peptides in and out of the CDK2/CA crystals was slow and in some cases, even after a period of three days of soaking in new peptide solution, the original peptide could still be observed to occupy the binding groove. However, occasional agitation of the crystallisation mixture during the three-day period led to complete ligand exchange. Solution studies for peptides binding to the CBG using a competitive binding assay (McInnes et al, 2003) demonstrated that equilibrium is reached within a few seconds. Buffer and salt conditions in these solution studies and in the crystal exchange experiments described here were very similar, apart from the high PEG concentrations in the crystal soaking experiments. It therefore seems that diffusion rates play a major role during ligand exchange in crystals. Fortuitously, the crystal system described has two ligand binding sites with different crystallographic environments, thereby leading to unambiguous results for the accessible site. The results with the cyclin-CDK2 system described herein suggest that the ligand exchange approach may be of general use in different protein systems where native protein crystals are not readily obtainable, or where protein-recognition sites are occupied by neighbouring molecules in the crystal lattice.

Interactions between Ligand Peptides and Cyclin A

The binding site for the CBM peptides is comprised of a shallow groove resulting from the exposed surface of the cyclin A α1, α3, and α4 helices. The residues lining the groove form a number of subsites including the LYY′ hydrophobic pocket (Met²¹⁰, Ile²¹³, Leu²¹⁴, Trp²¹⁷, Leu²⁵³), the arginine site (RX; Glu²²⁰), and a secondary lipophilic pocket we call the alanine pocket (Val²²¹, Ile²⁸¹). In addition, binding is also facilitated by a number of H-bond interactions with the peptide backbone, including a pair of interactions with the side-chain carboxamide of Gln²⁵⁴. Despite the well-documented observation that the CDK2 kinase subunit of the CDK2/CA complex undergoes significant conformational changes upon cyclin binding and T-loop phosphorylation, the cyclin subunit structure is essentially the same in the free and complexed forms (Brown et al., 1995). In our peptide complex structures, most of the cyclin A binding groove (CBG) atoms are rigid and their average B factors do not change upon ligand binding. This was confirmed by comparison of the temperature factors of the CBG atoms with the rest of the cyclin A chain, as well as by comparison of the unliganded and peptide-liganded CDK2/CA structures. All main chain atoms of the residues in the groove have low B factors in both the peptide-bound and unliganded structures, although there are some significant differences in the side chains of the hydrophilic residues of the binding groove.

All residues in all of the peptide ligands complexes presented here are well defined by electron density. The electron density difference maps of the peptide complexes are shown in FIG. 3. As mentioned, two CDK2/CA complexes exist in the asymmetric unit of our crystal form. The differences between the two copies of peptide 2 structures bound in the different CBGs are illustrated in FIG. 3a. The RMS deviation for all atoms (50) after superimposition was 0.163 Å. Arg¹, Leu³, and Phe⁵ make similar contacts in both cases, with some variation for Arg². These variations should be considered in the context of the flexible Glu²²⁰ receptor side chain and are discussed in more detail below. Ile⁴ does not make contact with the binding groove and is restrained only through intramolecular contacts with Arg². Arg² itself participates in charge-charge interactions with cyclin A, as well as some weak contacts with the neighbouring CDK2 molecules in the case of the partially occluded CBG.

An overlay of all the solved peptide structures is shown in FIG. 3b. Although the peptides bind in superficially similar modes, significant differences in inhibitory potency, and hence binding affinity, were observed between the peptides. These differences can be explained after detailed examination of the specific interactions formed or lost in each case, and are summarised below.

Peptide Potency and Design

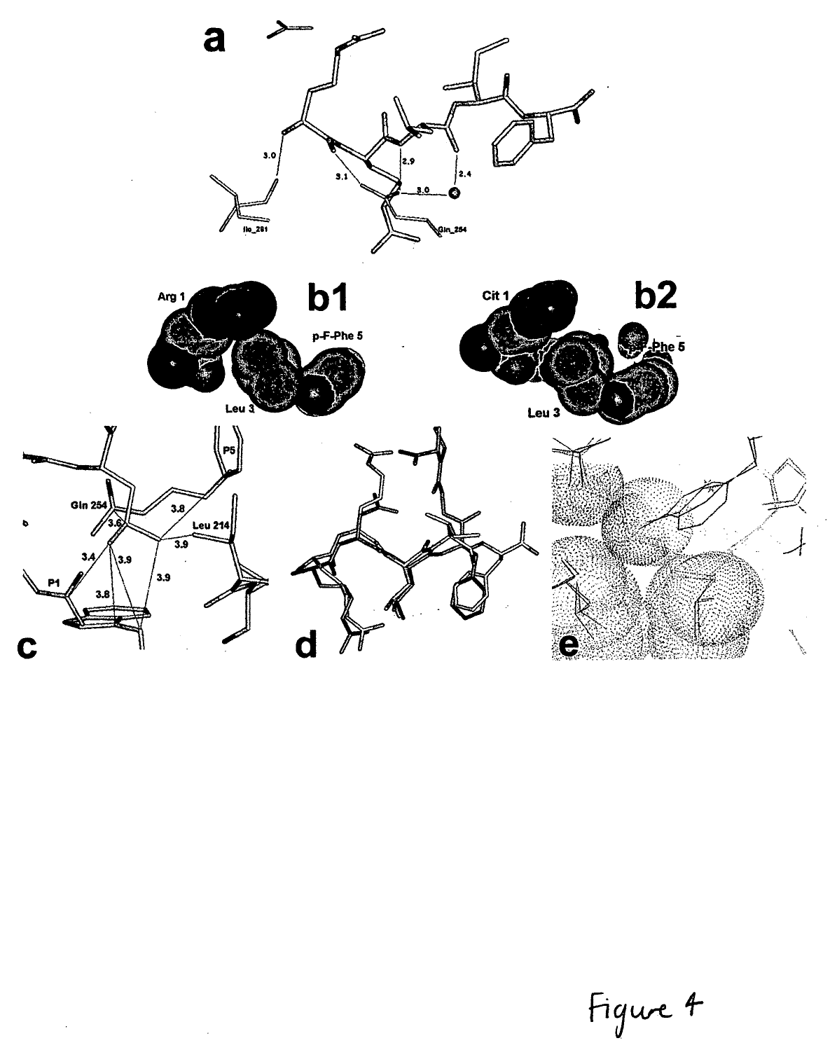

Analysis of peptide 2 complexed with cyclin A shows that the bound conformation is stabilised through a number of contacts with the CBG, involving the side chains of Arg¹, Leu³, and Phe⁵, as well as the peptide backbone. (FIG. 4a). The latter include the Arg¹ terminal NH₂, Arg¹ CO, and Leu³ NH groups. Judging from the interatomic distances, however, none of these form strong H-bonds. Additionally, a number of intramolecular interactions involving Arg¹, Leu³, and Phe⁵ are observed. It is clear from the peptide crystal structures that residues P¹, P³, and P⁵ will be the main potency determinants, while P² and P⁴, whose side chains project away from the binding groove, will be less important. Furthermore, the P¹, P³, and P⁵ side chains appear to form an intramolecular network of interactions. Thus the side chains of Arg¹ and Phe⁵ interact with the cyclin groove in such a way as to trap between them the Leu³ side chain (FIG. 4b1). The relative contribution to binding of each residue in the peptides can be judged from the activity summary in Table 2. It should be kept in mind when analysing the potency data that the effect of any one mutation will be more profound in the context of a shorter than of a longer peptide.

P¹ Site

A key interaction in the CBM, and a requirement for good inhibitory potency of relevant peptides, is the ion pair between Glu²²⁰ and Arg¹. The importance of Arg in this position was also borne out in previous structures (Lowe et al., 2002) and mutation of Arg¹ always results in a decrease in biochemical inhibitory activity (McInnes et al., 2003). Even the isosteric substitution of Arg¹ with Cit¹, i.e. replacement of the guanidine —NHC═NH(NH₂) with an urea —NHC═O(NH₂), leads to a significant decrease in potency. Replacement of the ω imino function in Arg with the carbonyl group in Cit results in a system with similar H-bonding ability but unlike the Arg guanidine, the Cit urea is not charged under physiological conditions and therefore does not participate in ion pair interactions. Analysis of the peptide 5 complex crystal structure shows that the distance between Cit¹ NH and the Glu²²⁰ side chain carbonyl O is 5.1 Å, whereas the corresponding distance in the peptide 4 structure is 4.5 Å. This difference effectively results in loss of the contacts between Glu²²⁰ and the Cit-containing peptide. A similar picture emerges from comparison of the average cyclin A Glu²²⁰ side chain atom B factors in both the peptide 5 and peptide 2 complex structures. In the latter structure this factor lies only 20.5% above the average for all main chain atoms, whereas in the unliganded and peptide 5-liganded complexes the value rises to 45 and 67%, respectively. This is a clear indication of large displacement of Glu²²⁰ side chain atoms when peptide 5 binds in the CBG.

Another factor contributing to the decrease in activity is the loss of intramolecular contacts with Leu³. The peptide 5 2Fo-1Fc map shows an absence of density for the Leu³ side chain beyond the Cβ atom, indicating free rotation about the Cα-Cβ bond. By contrast, all atoms of the Leu³ side chain are clearly visible in the electron density difference maps for the peptide 2 and peptide 4 structures (FIG. 3). Apart from these differences at P¹ and P³, the positions of the remaining atoms are similar, including the P⁵ side chain conformation.

The effect of acetylation of the N-terminal nitrogen was also investigated. In the peptide 2 complex structure, the peptide amino terminus forms a H-bond (3 Å) with the carbonyl of Ile²⁸¹ and a similar H-bond is also observed in peptide 4 and peptide 3 structures. The weakening of this H-bond (3.4 Å) upon acetylation of the peptide N-terminus is consistent with the reduced biological activity of acetylated peptides (Table 2). An additional interesting observation relating to this involves the Arg¹ side chain, which cannot make the same contacts in the acetylated and non-acetylated peptides. In the acetylated peptide a series of contacts are observed that are absent in peptides with free N-termini and involve the acetyl O, a water molecule, the Glu²²⁰ side chain, as well as the NH group of the Arg¹ side chain.

P² Site

Analysis of the peptide activity data for different amino acid residues in this position suggests that it is comparatively tolerant of modification. This finding is supported by the complex structures, which do not show any strong interactions between the P² side chain and the protein. In contrast to P¹, replacement of the charged side chain at P² has comparatively little effect on biological activity, as is apparent from the Arg²-Cit mutation. Inclusion of certain uncharged residues at P² is also tolerated to some extent, e.g. Gln (Table 2) or Ser (McInnes et al., 2003). The observed 3-fold reduction in potency, however, is consistent with the decrease in the Coulombic interaction energies calculated upon substitution of the Arg² with Asn.

P³ Site

The necessity of a hydrophobic side chain at this position, as indicated by the activity data, is obvious from all structures reported here. The Leu³ side chain participates in a network of van der Waals contacts with Gln²⁵⁴, Trp²¹⁷, and Leu²¹⁴ of cyclin A, in addition to several intramolecular contacts with P¹ and P⁵ (FIGS. 4b & c ). Inspection of the complex structures and molecular modelling suggest that the Leu side chain is the optimum size for this position. Mutation of Ile is tolerated, but results in bad contacts and mutation to Ala leads to significant loss of potency. The Leu³ side chain forms the core of the binding surface for all of the peptides investigated and a residue with a side chain of lesser bulk in this position would allow the side chains at P¹ and P⁵ to move freely. This in turn would result in disruption of the bound conformation, similar to the effect described above for P¹, but with a more profound result since now both neighbouring side chains and their protein contacts would be affected.

P⁴ Site

The major role of this residue (as encountered in the p21^{WAF1} and p53 CBMs), is to act as a linker between the P³ and P⁵ hydrophobic groups. Incorporation of a linker is favourable for binding and in the peptides tested improves potency relative to the p27^{KIP1} CBM, where the P⁴ residue is absent (Leu³-Phe⁵-Gly⁶). The extra 3 atoms of the Ile⁴ residue in the backbone structure allow P⁵ to adopt a more favourable position in the groove and make more complementary contacts with the hydrophobic pocket (FIG. 4d). Also as is discussed in the following section, the linker residue allows a more favourable intramolecular peptide conformation of the Leu³ and Phe⁵ residues. In addition, the Ile⁴ spacing residue allows the P³ carbonyl in peptide 2 to form a strong water-mediated H-bond (2.4 Å) with the Gln²⁵⁴ side chain (FIG. 4a). In contrast, this carbonyl in the p27^{KIP1} complex structure is rotated by almost 180 ° (FIG. 4d) and consequently this H-bond is not present. These observations are backed up by the biochemical data since removal of Ile³ results in 12-fold lower potency (Table 2). Furthermore, comparison of the structural data confirms that due to the different conformations of the Phe⁵ side chain, the p-F-Phe phenyl ring substitution is less effective in the context of the Leu-Phe-Gly sequence; the same modification of the aromatic side chain of Phe⁵ results in considerable potency gains in the Leu-X-Phe motif (Table 2). In all of the structures reported here, the P⁴ side chain does not make any contacts with cyclin A. Despite this observation, peptide potency does depend to some extent on the nature of P4 . Aβ-substituted side chain is preferred, presumably as it favourably reduces peptide flexibility in the unbound state.

P⁵ Site

This site was probed with a variety of residues leading to the conclusion that certain aromatic side chains are optimal (McInnes et al., 2003). The most potent Phe derivatives were found to be para-substituted (Table 2). The crystal structures of three peptides in which P⁵ is m-Cl-Phe or p-F-Phe in complex with the cyclin A groove demonstrate that P⁵ always occupies the same site (FIG. 4e). The P⁵ side chain in peptide 3 adopts a different conformation to that in the other structures (FIG. 3b). Here the meta-chloro group forces the Phe⁵ ring into a different conformation. As a consequence, it forms bad contacts with Met²¹⁰; these are offset, however, by the more favourable interactions of the chloro group with the base of the lipophilic pocket. The observed contacts with this pocket are so specific that the chlorine and fluorine van der Waals surfaces coincide very closely in the two superimposed peptide structures, despite the differing positions of the aromatic rings (FIG. 4e). This observation reinforces the optimal substitution position of the P⁵ phenyl ring as being para, a result reflected by the peptide activity data (Table 2).

Computational Analysis

In an attempt to understand better the observed differences in affinity for p21^{WAF1} versus p27^{KIP1}-derived CBM peptides, intermolecular non-bonded energetics were calculated using the X-ray complex structures of the peptide 2 (p21^{WAF1}) and H-Arg-Asn-Leu-Phe-Gly-OH 4 (p27^{KIP1}; 1H27.pdb) (SEQ. ID No: 9) peptide (Lowe et al., 2002) (Table 3). The results help to explain the individual contributions for the p21^{WAF1} and p27^{KIP1} CBM peptides in terms of binding energy and indicate that a major contribution to the observed difference in affinity is due to the presence of a second charged Arg at P² in p21^{WAF1}. Although this residue does not appear to make specific protein contacts, it does contribute significantly via Coulombic energy due to its proximity with Asp²⁸². In addition, there also appears to be considerable contribution to binding from the surrounding solvent-mediated H-bonds. Whereas the p27^{KIP1} peptide structure (Russo et al., 1996) contains a single water molecule interacting with the peptide, we refined our structures to include as many crystallographically defined water molecules as could be found, especially in the binding groove, for the purpose of using these in drug design. Specifically, the energetic analysis suggests that a water-mediated H-bond from the Leu³ carbonyl to the Gln²⁵⁴ side chain provides 14 kcal/mol to the binding energy of the p21^{WAF1} sequence. We also carried out energy minimisation calculations on the bound conformations for peptide 2 and the H-Arg-Arg-Leu-Phe-NH₂ (SEQ. ID No: 20) peptide in isolation from cyclin A. The energy difference between the minimised peptide conformation and that observed when bound to the CBG is an indication of the energetic penalty a peptide has to pay during binding. Since peptide 2 has to form non-optimal intramolecular contacts and torsion angles in order to form complementarity with the cyclin groove, there is an unfavourable enthalpic contribution to the overall free energy. This was shown from the observation that the energy difference between the bound and minimised bound peptides was much greater for the p27^{KIP1} (54 kcal/mol) versus the p21^{WAF1} sequence (17 kcal/mol). As the binding energetics are comparable for peptide 2 and H-Arg-Arg Leu-Phe-NH₂, (SEQ. ID No: 13) this factor most likely explains the more than 10-fold potency decrease for the latter peptide. Those two peptides adopt a very similar conformation for the first three residues up to Leu³ (FIG. 4d). The main differences between the bound conformations are observed after Leu³. These differences are the result of non-ideal values for the intramolecular contacts in the p27^{KIP1} versus the p21^{WAF1} bound peptides, which also reflects the less favourable (φ, χ₁, χ₂ and ω torsion angles of Leu³ and Phe⁴ for the p27^{KIP1} peptide (Table 3). While these energetic calculations are obviously approximate, they nonetheless provide insight into the potency differences between cyclin groove inhibitor peptides.

Assay

A further aspect of the invention relates to an assay for screening one or more ligands capable of binding to the cyclin binding groove of CDK2/cyclin A, said assay comprising contacting a crystal as defined above, or obtainable by the above-described method, with a candidate ligand, L″, and detecting any change in the interaction between ligand L and CDK2/cyclin A.

In a preferred embodiment, the assay is a competitive binding assay.

Preferably, any change in the interaction between ligand L and CDK2/cyclin A can be monitored by detecting the displacement of ligand L from the CDK2/cyclin A/L complex and/or by monitoring formation of a complex comprising CDK2/cyclin A and candidate ligand L″.

More preferably, the assay is used to screen for a ligand useful as an inhibitor of CDK2/cyclin A.

Preferably, the assay is used to screen for a ligand useful in the prevention and/or treatment of proliferative disorder such as cancers, leukaemias and other disorders associated with uncontrolled cellular proliferation such as psoriasis and restenosis. Preferably, the proliferative disorder is a cancer or leukaemia. The term proliferative disorder is used herein in a broad sense to include any disorder that requires control of the cell cycle, for example cardiovascular disorders such as restenosis, cardiomyopathy and myocardial infarction, auto-immune disorders such as glomerulonephritis and rheumatoid arthritis, dermatological disorders such as psoriasis, anti-inflammatory, anti-fungal, antiparasitic disorders such as malaria, emphysema, alopecia, and chronic obstructive pulmonary disorder. In these disorders, the ligands of the present invention may induce apoptosis or maintain stasis within the desired cells as required.

The ligands of the invention may inhibit any of the steps or stages in the cell cycle, for example, formation of the nuclear envelope, exit from the quiescent phase of the cell cycle (G0), G1 progression, chromosome decondensation, nuclear envelope breakdown, START, initiation of DNA replication, progression of DNA replication, termination of DNA replication, centrosome duplication, G2 progression, activation of mitotic or meiotic functions, chromosome condensation, centrosome separation, microtubule nucleation, spindle formation and function, interactions with microtubule motor proteins, chromatid separation and segregation, inactivation of mitotic functions, formation of contractile ring, and cytokinesis functions. In particular, the compounds of the invention may influence certain gene functions such as chromatin binding, formation of replication complexes, replication licensing, phosphorylation or other secondary modification activity, proteolytic degradation, microtubule binding, actin binding, septin binding, microtubule organising centre nucleation activity and binding to components of cell cycle signalling pathways.

More preferably, the assay is used to screen for a ligand useful in the prevention and/or treatment of cancer, leukemia, a viral disorder, stroke, glomerulonephritis, alopecia, diabetes, a CNS disorder, rheumatoid arthritis, psoriasis or chronic obstructive pulmonary disorder.

Ligands which are identified using the crystal of the present invention can be screened in assays such as those well known in the art. Screening can be, for example, in vitro, in cell culture, and/or in vivo. Biological screening assays preferably centre on activity-based response models, binding assays (which measure how well a compound binds to the receptor), and bacterial, yeast and animal cell lines (which measure the biological effect of a compound in a cell). The assays can be automated for high capacity-high throughput screening (HTS) in which large numbers of compounds can be tested to identify compounds with the desired activity. The biological assay, may also be an assay for the ligand binding activity of a compound that selectively binds to the LBD compared to other nuclear receptors.

Another aspect of the invention relates to a process comprising the steps of:
(a) performing the above-described assay;
(b) identifying one or more ligands capable of binding to the cyclin binding groove; and
(c) preparing a quantity of said one or more ligands.

Another aspect relates to a process comprising the steps of:
(a) performing the above-described assay;
(b) identifying one or more ligands capable of binding to the cyclin binding groove; and
(c) preparing a pharmaceutical composition comprising said one or more ligands.

Another aspect of the invention relates to a process comprising the steps of:
(a) performing the above-described assay;
(b) identifying one or more ligands capable of binding to the cyclin binding groove;
(c) modifying said one or more ligands capable of binding to the cyclin binding groove;
(d) performing the above-described assay;
(e) optionally preparing a pharmaceutical composition comprising said one or more ligands.

Yet another aspect of the invention relates to a ligand identified by the above-described assay. Preferably, the identified ligand may act as a ligand model (for example, a template) for the development of other compounds.

The crystal structure of the present invention can be used to generate a structural model such as a three dimensional (3D) structural model (or a representation thereof). Alternatively, the crystal structure may be used to generate a computer model for the structure.

Thus, for example, the structural co-ordinates provided in the crystal structure and/or model structure may comprise the amino acid residues of the cyclin binding groove, or a portion thereof, or a homologue thereof useful in the modelling and design of test compounds or further ligands capable of binding to the cyclin binding groove.

As used herein the term “three dimensional model” includes both crystal structures as determined by X-ray diffraction analysis, solution structures determined by nuclear magnetic resonance spectroscopy as well as computer generated models. Such computer generated models may be created on the basis of a physically determined structure of a peptide of the present invention bound to cyclin A or on the basis of the known crystal structure of cyclin A, modified (by the constraints provided by the software) to accommodate a peptide of formula I. Suitable software suitable of the generation of such computer generated three dimensional models include AFFINITY, CATALYST and LUDI (Molecular Simulations, Inc.).

Such three dimensional models may be used in a program of rational drug design to generate further candidate compounds that will bind to cyclin A. As used herein the term “rational drug design” is used to signify the process wherein structural information about a ligand-receptor interaction is used to design and propose modified ligand candidate compounds possessing improved fit with the receptor site in terms of geometry and chemical complementarity and hence improved biological and pharmaceutical properties, such properties including, e.g., increased receptor affinity (potency) and simplified chemical structure. Such candidate compounds may be further compounds or synthetic organic molecules.

As used herein, the term “modelling” includes the quantitative and qualitative analysis of molecular structure and/or function based on atomic structural information and interaction models. The term “modelling” includes conventional numeric-based molecular dynamic and energy minimization models, interactive computer graphic models, modified molecular mechanics models, distance geometry and other structure-based constraint models.

In another aspect of the present invention, the structural coordinates comprising the cyclin binding groove, or portion thereof, and the ligand bound thereto, may be applied to a model screening system.

As used herein, the term “model screening system” may be a solid 3D screening system or a computational screening system. Using this model, test compounds may be modelled which fit spatially into the cyclin binding groove.

In one preferred aspect, the test compounds are positioned in the cyclin binding groove through computational docking.

In another preferred aspect, the test compounds are positioned in the cyclin binding groove through manual docking.

As used herein, the term “fits spatially” means that the three-dimensional structure of a ligand is accommodated geometrically in the cyclin binding groove.

Preferably, modelling is performed using a computer and may be further optimized using known methods. This is called modelling optimisation.

Overlays and super positioning with a three dimensional model of cyclin binding groove, and/or a portion thereof, can also be used for modelling optimisation. Additionally, alignment and/or modelling can be used as a guide for the placement of mutations on the cyclin binding groove to characterise the nature of the site in the context of a cell.

The three dimensional structure of a new crystal may be modelled using molecular replacement. The term “molecular replacement” refers to a method that involves generating a preliminary model of a molecule or complex whose structure coordinates are unknown, by orienting and positioning a molecule whose structure coordinates are known within the unit cell of the unknown crystal, so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. Lattman, E., “Use of the Rotation and Translation Functions”, in Methods in Enzymology, 115, pp. 55-77 (1985); M. G. Rossmann, ed., “The Molecular Replacement Method”, Int. Sci. Rev. Ser., No. 13, Gordon & Breach, New York, (1972).

Other molecular modelling techniques may also be employed in accordance with this invention. See, e.g., Cohen, N. C. et al, “Molecular Modelling Software and Methods for Medicinal Chemistry”, J. Med. Chem., 33, pp. 883-894 (1990). See also, Navia, M. A. and M. A. Murcko, “The Use of Structural Information in Drug Design”, Current Opinions in Structural Biology, 2, pp. 202-210 (1992).

Using the structure coordinates of the crystal complexes provided by this invention, molecular replacement may be used to determine the structure coordinates of a crystalline mutant or homologue of the cyclin binding groove of CDK2/CA or of a related protein.

As used herein, the term “mutant” refers to any organism that has undergone mutation or that carries a mutant gene that is expressed in the phenotype of that organism. A mutation may arise due to a substitution of one nucleotide for another or from a deletion of a nucleotide or an insertion of a nucleotide relative to a referenced wild type sequence. These single nucleotide variations are sometimes referred to as single nucleotide polymorphisms (SNPs). Some SNPs may occur in protein-coding sequences, in which case, one of the polymorphic forms may give rise to the expression of a defective or other variant protein and, potentially, a genetic disease. Other SNPs may occur in noncoding regions. Some of these polymorphisms may also result in defective protein expression (e.g., as a result of defective splicing). Other SNPs may have no phenotypic effects.

Another aspect relates to the use of a ligand identified by the above-described assay in the preparation of a medicament for treating a proliferative disorder.

Another aspect relates to the use of a ligand identifed by the above-described assay in the preparation of a medicament for treating cancer, leukemia, a viral disorder, stroke, glomerulonephritis, alopecia, diabetes, a CNS disorder, rheumatoid arthritis, psoriasis or chronic obstructive pulmonary disorder.

Pharmaceutical Compositions

Another aspect of the invention relates to a pharmaceutical composition comprising a ligand identified by the above assay admixed with one or more pharmaceutically acceptable diluents, excipients or carriers. Even though the ligands (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Salts/Esters

The ligands identified by the assay of the invention can be present as salts or esters, in particular pharmaceutically acceptable salts or esters.

Pharmaceutically acceptable salts of the ligands identified by the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen). Enantiomers/Tautomers

In all aspects of the present invention previously discussed, the invention includes, where appropriate all enantiomers and tautomers of ligands identified by the assay of the invention. The man skilled in the art will recognise compounds that possess an optical properties (one or more chiral carbon atoms) or tautomeric characteristics. The corresponding enantiomers and/or tautomers may be isolated/prepared by methods known in the art.

Stereo and Geometric Isomers

Some of the ligands of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those agents, and mixtures thereof. The terms used in the claims encompass these forms, provided said forms retain the appropriate functional activity (though not necessarily to the same degree).

The present invention also includes all suitable isotopic variations of the ligand or pharmaceutically acceptable salts thereof. An isotopic variation of a ligand of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts threeof include isotopes of hydrogen, carbon, nitrogen, oxygen, phosphorus, sulphur, flourine and chlorine such as, ²H, ³H, ¹³C , ¹⁴C , ¹⁵N, ¹⁷O, ¹⁸O, ³¹P, ³²P, ³⁵S, ¹⁸F, and ³⁶Cl respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

Solvates

The present invention also includes the use of solvate forms of the ligands of the present invention. The terms used in the claims encompass these forms.

Polymers

The invention furthermore relates to the ligands of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

Prodrugs

The invention further includes the ligands of the present invention in prodrug form. Such prodrugs are generally compounds wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

By way of summary, the technique of ligand exchange described herein is an effective way of obtaining crystallographic information on series of compounds without having to depend on successful co-crystallisation. As has been highlighted, many proteins do not yield crystals in the absence of suitable ligands. In the case studied, the use of a suitable starting ligand allowed the formation of crystals, which were subsequently employed to provide structural information for numerous ligands. The results reported here suggest that the ligand exchange approach may be of general use in different protein systems where native protein crystals are not readily obtainable, for example when contacts of surface-bound ligands with neighbouring molecules are required for crystallisation.

The peptides contain residues that are not directly involved in binding but act as spacers and help to orient those groups involved in groove recognition. The charged amino terminus and side chain functions at P¹ (Arg) are of prime importance for potency in the context of pentapeptide inhibitors. Replacement of the charged guanidine function with the uncharged isosteric urea group (Cit) and acetylation of the N-terminus lead to ca. 36-fold and 3.8-fold potency reductions, respectively. The importance of the P¹ charged side chain is less pronounced in the octapeptide inhibitor series (McInnes et al., 2003), where the loss of the charge-charge interaction is partially offset by other favourable interactions not available in the pentapeptides. A second charged residue present at P² in CBMs can be eliminated more readily, due to the lesser contribution to binding. Although the side chain of Arg² does not form direct contacts with the cyclin groove, it clearly participates in long-range electrostatic interactions. Arg at P² can be effectively replaced e.g. with Cit or Gln, and with other residues capable of both donating and accepting H-bonds. The Leu residue at P³ is invariant in all known CBMs and our results confirm that the Leu side chain optimally occupies the hydrophobic pocket at that site. The P⁴ residue serves predominantly as a linker and a steric constraint. As it does not form any direct interactions with the protein, it is a potential candidate for modifications designed to improve the physicochemical characteristics of future inhibitors. Finally, P⁵ is preferably an aromatic residue with a halogen-substituted phenyl group. The crystal structures and potency data indicate that occupying the volume filled by the halogen atoms makes important contributions to binding and hence should be accounted for in ligand design. Molecular modelling shows that there is sufficient space around this ring in the hydrophobic groove pocket to accommodate additional substituents, which could further increase the buried surface and hence the potency.

Overall it has been shown that peptide potency can be regained after truncation of the p21^{WAF1} octapeptide and that a molecule with a lower net charge can be obtained through incorporation of unnatural amino acids. The structural basis for these improvements was rationalised and suggests that the development of small molecule inhibitors of the CDK2/CA via the substrate-binding site is feasible. The important features of both the binding site and bound peptides incorporating non-natural residues found during this work should facilitate the design of more potent and less peptidic inhibitors.

The present invention is further described by way of Example and with reference to the following Figures, wherein:

### EXAMPLES

Expression and Purification of CDK2, Cyclin A, and CDK2/CA Complex

Human recombinant CDK2 was expressed and purified as described (Wu et al., 2003). Human recombinant cyclin A2 (fragment encompassing residues 173-432) was expressed in Escherichia coli BL21 (DE3) using PET expression vectors. BL21 (DE3) was grown at 37° C. with shaking (200 r.p.m) to mid-log phase (A₆₀₀ₙₘ˜0.6). Expression was induced by the addition of IPTG at a final concentration of 1 mM and the culture was incubated for a further 3 h. Bacteria were harvested by centrifugation, and the cell pellet was resuspended in buffer A (25 mM Tris pH 8.0, 5 mM DTT, 1 mM PMSF, 1 mM EDTA, 1 mM benzamidine, and protease inhibitor cocktail). After sonication the lysate was clarified by centrifugation for 30 min at 15,000 g and 4° C. The supernatant was passed through a DEAE-Sepharose column (pre-equilibrated with buffer B (25 mM Tris pH 8.0, 2 mM DTT, 1 mM PMSF, 1 mM EDTA, 1 mM benzamidine, and 10 mM NaCl). After washing, bound protein was eluted with an ascending concentration gradient of NaCl in buffer B. The fractions containing cyclin A (typically eluted at 300-400 mM NaCl) were pooled, diluted (5 times in buffer A without protease inhibitor cocktail), and loaded on to a pre-equilibrated (with buffer B) SP-Sepharose column. After washing, bound protein was eluted with a NaCl gradient in buffer B. The fractions containing cyclin A (typically eluted at 300-400 cmM NaCl) were pooled, diluted (5 times in buffer A without protease inhibitor cocktail) and loaded on to a pre-equilibrated (with buffer B) High-Trap Q column. After washing, bound protein was eluted with a NaCl gradient in buffer B. Pooled fractions containing pure cyclin A were mixed in a 1:1 molar ratio with CDK2 and were concentrated before loading on to a pre-equilibrated (with 50 mM Tris pH 8.2, 100 mM NaCl, 1 mM EDTA, 1 mM DTT) Superdex 75 SE column. Eluted fractions containing stoichiometric mixtures of CDK2 and cyclin A were used for crystallisation after concentration by ultrafiltration (Amicon concentration unit).

Crystallisation and Structure Determination

The CDK2/CA/peptide crystals were grown by vapour diffusion using the hanging drop method. The peptides H-Arg-Arg-Leu-Ile-Phe-NH₂ (SEQ. ID No: 2) and H-Arg-Arg-Leu-Ile-(p-F-Phe)-NH₂ (SEQ. ID No: 4) were co-crystallised with CDK2/CA.

Typically, a 2-μL solution of CDK2/cyclin A (7-8 mg/mL in 40 mM HEPES pH 7.0, 200 mM NaCl, 5 mM DTT) and containing a 5-fold molar excess of peptide was mixed with 2 μL of well solution. The precipitant solution contained 18% PEG-3,350 and 100 mM trisodium citrate. Crystals were obtained after 1-2 weeks at 17° C. CDK2/CA complexes with Ac-Arg-Arg-Leu-Asn-(m-Cl-Phe)-NH₂, (SEQ. ID No: 3) H-Cit-Cit-Leu-Ile-(p-F-Phe)-NH₂, (SEQ. ID No: 5) and H-Ala-Ala-Abu-Arg-Ser-Leu-Ile-(p-F-Phe)-NH₂ (SEQ. ID No: 1) were prepared by a ligand exchange technique using CDK2/CA/H-Arg-Arg-Leu-Ile-Phe-NH₂ (SEQ. ID No: 2) complex crystals. Initially the exchange technique was carried out in two steps. First, H-Arg-Arg-Leu-Ile-Phe-NH₂ peptide (SEQ. ID No: 2) was eluted from the crystal after soaking in a solution containing 18% PEG-3,350 and 100 mM trisodium citrate. The unliganded crystal was then soaked in 18% PEG-3,350, 100 vmM trisodium citrate and 1 mM of one of the above peptide. Later experiments showed that elution of H-Arg-Arg-Leu-Ile-Phe-NH₂ (SEQ. ID No: 2) and soaking of a new peptide could be performed simultaneously. The final protocol consisted of soaking of CDK2/ CA/peptide A crystals in mother liquor plus 1 mM peptide B for three days. The soaking solution (˜10 μL) was replaced and the mixture stirred regularly during the soaking period. Under the experimental conditions used here, one crystal was soaked in a 10-μL drop of a 1 mM exchange peptide solution. This corresponds to less than 5 pmol of CDK2/CA mixed with 10 nmol of exchange peptide. Assuming a simple bimolecular equilibrium, the dissociation constant (K_{c}) between the ligand L and the protein P crystal is given by: K_{c}=([P_{unbound}] [L_{unbound}])/[PL]. This can be simply related to crystallographic (Wu et al., 2001) occupancy (Q) by K_{c}=(1−Q)×[L_{free}]/Q. Thus for K_{c} values between 1 and 100 μM, and assuming unrestricted access to the cyclin ligand binding sites in the crystal, crystallographic occupancies would be expected to be greater than 90%.

Crystals of about 0.05×0.1×0.1 mm were mounted in 0.05-0.1 mm cryo-loops (Hampton Research). The crystals were immersed briefly in a cryoprotectant (28% PEG-3,350 and 0.1 M trisodium citrate) and then flash-frozen in liquid nitrogen. Data were collected at the Daresbury (UK) and Grenoble (France) synchrotron facilities. Data processing was carried out using the program DENZO and SCALEPACK (Otwinowski and Minor, 1997), or MOSFLM (Leslie, 1992) and SCALA (Evans, 1993) from the CCP4 program suite (1994). Data often showed signs of crystal radiation damage. The structures were solved by molecular replacement using MOLREP (Vagin and Teplyakov, 1997) and PDB entries 1 HCL or 1 CKP as the search model. ARP/wARP (Lamzin and Wilson, 1997) was used for initial density interpretation and the addition of water molecules. REFMAC (Murshudov et al., 1997) was used for structural refinement. A number of rounds of refinement and model building with the program Quanta (Accelrys, San Diego, USA) were carried out.

Computational Chemistry

Calculation of protein ligand interaction energies for the cyclin A inhibitors was performed using the program CDISCOVER after addition of hydrogens and steepest-descent minimisation, using the modelling package InsightII (Accelrys, San Diego, USA).

Peptide Synthesis

Peptides were prepared, purified, and characterised using procedures as previously described (Zheleva et al., 2002).

Biochemical Assays

These were carried out as previously described (Atkinson et al., 2002; McInnes et al., 2003; Zheleva et al., 2002).

Accessions Numbers

Structures have been deposited in the Protein Data Bank under ID codes 1OKU, 1OKV, 1OKW, 1OL2 and 1OL1 for the CDK2/CA complexes with peptides 1-5.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

### REFERENCES

Anon. (1994). Number 4 Collaborative Computional Project: The CCP4 suite: programs for protein crystallography. Acta Crystallogr. D50, 760-763.
Atkinson, G. E., Cowan, A., McInnes, C., Zheleva, D. I., Fischer, P. M., and Chan, W. C. (2002). Peptide Inhibitors of CDK2-cyclin A that Target the Cyclin Recruitment-Site:Structural Variants of the C-Terminal Phe. Bioorg. Med. Chem. Lett. 12, 2501-2505.
Ball, K. L., Lain, S., Fahraeus, R., Smythe, C., and Lane, D. P. (1996). Cell-cycle arrest and inhibition of CDK4 activity by small peptides based on the carboxy-terminal domain of p21WAF1. Curr. Biol. 7, 71-80.
Bonfanti, M., Taverna, S., Salmona, M., D'Incalci, M., and Broggini, M. (1997). p21^{WAF1}-derived peptides linked to an internalization peptide inhibit human cancer cell growth. Cancer Res. 57, 1442-1446.
Brown, N. R., Noble, M. E., Endicott, J. A., Garman, E. F., Wakatsuki, S., Mitchell, E., Rasmussen, B., Hunt, T., and Johnson, L. N. (1995). The crystal structure of cyclin A. Structure 3, 1235-1247.
Brown, N. R., Noble, M. E., Endicott, J. A., and Johnson, L. N. (1999). The structural basis for specificity of substrate and recruitment peptides for cyclin-dependent kinases. Nat. Cell Biol. 1, 438-443.
Chen, J., Saha, P., Kombluth, S., Dynlacht, B. D., and Dutta, A. (1996). Cyclin binding motifs are essential for the function of p21CIP1. Mol. Cell. Biol. 16, 4673-4682.
Chen, Y.-N. P., Sharma, S. K., Ramsey, T. M., Jiang, L., Martin, M. S., Baker, K., Adams, P. D., Bair, K. W., and Kaelin, W. G. (1999). Selective killing of transformed cells by cyclin/cyclin-dependent kinase 2 antagonists. Proc. Natl. Acad. Sci. USA 96, 4325-4329.
Evans, R. R. (1993). Data reduction. In Proceedings of CCP4 Study Weekend, 1993, on Data Collection & Processing, L. Sawyer, N. Isaacs, and S. Bailey, eds. (Science and Engineering Research Council UK, Daresbury Laboratory, Warrington), pp. 114-122.
Fischer, P. M. (2001). Recent advances and new directions in the discovery and development of cyclin-dependent kinase inhibitors. Curr. Opin. Drug Disc. Dev. 4, 623-634.
Fischer, P. M., Endicott, J., and Meijer, L. (2003). Cyclin-dependent kinase inhibitors. In Progress in Cell Cycle Research, L. Meijer, A. Jézéquel, and M. Roberge, eds. (Roscoff, France, Editions de la Station Biologique de Roscoff), pp. 235-248.
Fischer, P. M., Krausz, E., and Lane, D. P. (2001). Cellular delivery of impermeable effector molecules in the form of conjugates with peptides capable of mediating membrane translocation. Bioconjugate Chem. 12, 825-841.
Fischer, P. M., and Lane, D. P. (2000). Inhibitors of cyclin-dependent kinases as anti-cancer therapeutics. Curr. Med. Chem. 7, 1213-1245.
Jeffrey, P. D., Russo, A. A., Polyak, K., Gibbs, E., Hurwitz, J., Massague, J., and Pavletich, N. P. (1995). Mechanism of CDK activation revealed by the structure of a cyclinA-CDK2 complex. Nature 376, 313-320.
Lamzin, V. S., and Wilson, K. S. (1997). Automated refinement for protein crystallography. Methods Enzymol. 277, 269-305.
Lees, J. A., and Weinberg, R. A. (1999). Tossing monkey wrenches into the clock: new ways of treating cancer. Proc. Natl. Acad. Sci. USA 96, 4421-4223.
Leslie, A. G. W. (1992). Recent changes to the MOSFLM package for processing film and image plate data. Joint CCP4+ESF-EAMCB Newsletter on Protein Crystallography 26.
Lowe, E. D., Tews, I., Cheng, K. Y., Brown, N. R., Gul, S., Noble, M. E. M., Gamblin, S. J., and Johnson, L. N. (2002). Specificity Determinants of Recruitment Peptides Bound to Phospho-CDK2/Cyclin A. Biochemistry 41, 15625 -15634.
McInnes, C., Andrews, M. J. I., Zheleva, D. I., Lane, D. P., and Fischer, P. M. (2003). Peptidomimetic design of CDK inhibitors targeting the recruitment site of the cyclin subunit., Curr. Med. Chem. Anti-Cancer Agents 3, 57-69.
Mendoza, N., Fong, S., Marsters, J., Koeppen, H., Schwall, R., and Wickramasinghe, D. (2003). Selective Cyclin-dependent Kinase 2/Cyclin A Antagonists that Differ from ATP Site Inhibitors Block Tumor Growth. Cancer Res. 63, 1020-1024.
Murshudov, G. N., Vagin, A. A., and Dodson, E. J. (1997). Refinement of macromolecular structures by the maximum-likelihood method. Acta Crystallogr. D53, 240-255.
Ortega, S., Malumbres, M., and Barbacid, M. (2002). Cell cycle and cancer: the G1 restriction point and the G1/S transition. Current Genomics 3, 245-263.
Otwinowski, Z., and Minor, W. (1997). Processing of x-ray diffraction data collected in oscillation mode. Methods Enzymol. 276, 307-326.
Russo, A. A., Jeffrey, P. D., Patten, A. K., Massague, J., and Pavletich, N. P. (1996). Crystal structure of the p27Kip1 cyclin-dependent-kinase inhibitor bound to the cyclin A-CDK2 complex. Nature 382, 325-331.
Schulman, B. A., Lindstrom, D. L., and Harlow, E. (1998). Substrate recruitment to cyclin-dependent kinase 2 by a multipurpose docking site on cyclin A. Proc. Natl. Acad. Sci. USA 95, 10453-10458.
Vagin, A., and Teplyakov, A. (1997). MOLREP: an automated program for molecular replacement. J. Appl. Crystallogr. 30, 1022 -1025.
Wu, S.-Y., Doman, J., Kontopidis, G., Taylor, P., and Walkinshaw, M. D. (2001). The first direct determination of a ligand binding constant in protein crystals. Angew. Chem., Int. Ed. 40, 582-586.
Wu, S. Y., McNae, I., Kontopidis, G., McClue, S. J., McInnes, C., Stewart, K. J., Wang, S., Zheleva, D. I., Marriage, H., Lane, D. P., Taylor, P., Fischer, P. M., and Walkinshaw, M. D. (2003). Discovery of a Novel Family of CDK Inhibitors with the Program LIDAEUS: Structural Basis for Ligand-Induced Disordering of the Activation Loop. Structure 11, 399-410.

Zheleva, D. I., McInnes, C., Gavine, A.-L., Zhelev, N. Z., Fischer, P. M., and Lane, D. P. (2002). Highly potent p21^{WAF1} derived peptide inhibitors of CDK-mediated pRb phosphorylation: delineation and structural insight into their interactions with cyclin A. J. Peptide Res. 60, 257-270.

TABLE 1
Crystallographic data and statistics

| | Peptide | | | | |
|---|---|---|---|---|---|
| Data collection | 1 | 2 | 3 | 4 | 5 |
| Space group | P2₁2₁2₁ | P2₁2₁2₁ | P2₁2₁2₁ | P2₁2₁2₁ | P2₁2₁2₁ |
| Unit cell a (Å) | 74.5 | 74.2 | 74.5 | 73.5 | 74.5 |
| b (Å) | 114.6 | 113.9 | 114.0 | 113.0 | 113.5 |
| c (Å) | 157.0 | 155.3 | 156.2 | 153.1 | 154.5 |
| Max. resolution (Å) | 2.9 | 2.4 | 2.5 | 2.6 | 2.9 |
| Observations | 430,926 | 563,579 | 553,149 | 574,133 | 648,091 |
| Unique reflections | 31,043 | 52,345 | 46,855 | 39,765 | 29,357 |
| Completeness (%) | 84.6 | 96.7 | 99.0 | 99.5 | 98.9 |
| R_{merge}¹ | 0.10 | 0.11 | 0.10 | 0.11 | 0.12 |
| Mean I/σ | 8.7 | 5.9 | 4.9 | 5.8 | 2.8 |
| Highest resolution bin | 2.97-2.9 | 2.46-2.4 | 2.64-2.5 | 2.74-2.6 | 3.06-2.9 |
| Mean I/σ (highest resolution bin) | 1.4 | 1.2 | 1.0 | 1.0 | 1.5 |
| R_{merge}¹ (highest resolution bin) | 0.59 | 0.65 | 0.47 | 0.62 | 0.46 |
| Refinement | | | | | |
| Protein atoms | 8,924 | 8,924 | 8,924 | 8,924 | 8,924 |
| Inhibitor atoms | 124 | 100 | 108 | 102 | 102 |
| Water | 196 | 746 | 539 | 383 | 75 |
| Reflections used in refinement | 24,032 | 47,769 | 44,775 | 36,002 | 27,895 |
| R_{factor} | 19.3 | 19.7 | 17.2 | 19.6 | 20.2 |
| R_{free} | 29.2 | 27.8 | 25.3 | 28.9 | 29.1 |
| Mean B-factor, protein (Å²) | 37.2 | 36.2 | 48.1 | 37.3 | 50.7 |
| Mean B-factor, ligands (Å²) | 47.5 | 44.2 | 54.3 | 48.5 | 66.3 |
| Mean B-factor, solvent (Å²) | 29.3 | 36.7 | 49.3 | 40.4 | 50.7 |

TABLE 2
Peptide structure-activity relationships summary

IC₅₀(μM)

| | | | | |
|---|---|---|---|---|
| Modifications | | | Competitive cyclin A | CDK2/cyclin A kinase |
| at position: | SEQ ID NO | Sequence | binding | activity |

| | | | | |
|---|---|---|---|---|
| 1 | SEQ ID NO: 21 | Ac-Arg-Arg-Leu-Asn-(p-F-Phe)-NH₂ | 2.0 | 12 |
| | SEQ ID NO: 4 | H-Arg-Arg-Leu-Asn-(p-F-Phe)-NH₂ | 0.53 | 7.2 |
| | SEQ ID NO: 22 | -H-Arg-Cit-Leu-Ile-(p-F-Phe)-NH₂ | 0.59 | 0.52 |
| | SEQ ID NO: 5 | -H-Cit-Cit-Leu-Ile-(p-F-Phe)-NH₂ * | 21 | 37 |
| 2 | SEQ ID NO: 23 | -H-Arg-Arg-Leu-Ala-(p-F-Phe)- NH₂ | 0.73 | 8.1 |
| | SEQ ID NO: 24 | -H-Arg-Cit-Leu-Ala-(p-F-Phe)- NH₂ | 2.6 | 24 |
| | SEQ ID NO: 25 | -H-Arg-Gln-Leu-Ile-(p-F-Phe)-NH₂ | 24 | 8.3 |
| | SEQ ID NO: 22 | -H-Arg-Cit-Leu-Ile-(p-F-Phe)-NH₂ | 0.59 | 0.52 |
| 3 | SEQ ID NO: 17 | H-His-Aia-Lys-Arg-Arg-Leu-Ile-Phe-NH₂ | 0.05 | 0.14 |
| | SEQ ID NO: 26 | H-His-Aia-Lys-Arg-Arg-Ala-Ile-Phe-NH₂ | 1.5 | >50 |
| 4 | SEQ ID NO: 5 | -H-Cit-Cit-Leu-Ile-(p-F-Phe)-NH₂ * | 21 | 37 |
| | SEQ ID NO: 27 | -H-Cit-Cit-Leu-Ala-(p-F-Phe)-NH₂ | 79 | 12 |
| | SEQ ID NO: 28 | -H-Cit-Cit-Leu-Asn-(p-F-Phe)-NH₂ | 19 | 37 |
| | SEQ ID NO: 2 | -H-Arg-Arg-Leu-Ile-Phe-NH₂ * | 0.68 | 7.7 |
| | SEQ ID NO: 20 | -H-Arg-Arg-Leu-—-Phe-NH₂ | 8.1 | >50 |
| | SEQ ID NO: 4 | -H-Arg-Arg-Leu-Asn-(p-F-Phe)-NH₂ | 0.53 | 7.2 |
| | SEQ ID NO: 29 | -H-Arg-Arg-Leu-—-(p-F-Phe)-NH₂ | 19 | 50 |
| 5 | SEQ ID NO: 30 | -Ac-Arg-Arg-Leu-Asn-Phe-NH₂ | 12 | >50 |
| | SEQ ID NO: 21 | -Ac-Arg-Arg-Leu-Asn-(p-F-Phe)-NH₂ | 2.0 | 12 |
| | SEQ ID NO: 3 | -Ac-Arg-Arg-Leu-Asn-(m-Cl-Phe)-NH₂ * | 5.6 | 31 |
| | SEQ ID NO: 31 | -Ac-Arg-Arg-Leu-Asn-(p-Cl-Phe)-NH₂ | 1.8 | 13 |

* Peptides with CDK2/CA complex structures.

TABLE 3
Comparison of binding energies and torsion angles of p21^{WAF1}- and p27^{KIP1}-derived CBM peptides.

| | | | | Torsion Angle (°) | | | | | |
|---|---|---|---|---|---|---|---|---|---|
| Interaction energy (kcal/mol) | | | | | Ideal | p21^{WAF1} | p21^{WAF1} | p27^{KIP1} | p27^{KIP1} |
| Peptide | 1 | 2 | 3 | Angle | values | Ile⁴ | Phe⁵ | Leu³ | Phe⁴ |
| Coulombic | −323 | −122 | −286 | φ | 65.3 | | 52 | | 86 |
| Van der Waals | −59 | −59 | −59 | χ₁ | 64.1 | | 72 | | 38 |
| Total | −383 | −181 | −345 | χ₂ | 177 | | 134 | | 81 |
| (cyclin A - peptide) | | | | | | | | | |
| Total | −410 | −194 | −356 | ω | 180 | 178 | — | 171 | — |
| (solvated cyclin A - peptide) | | | | | | | | | |
| H₂O-bridged H-bond* | −14 | | | | | | | | |
| Individual residues: | | | | | | | | | |
| P¹ | −107 | −104 | −104 | | | | | | |
| P² | −66 | −5.5 | −50 | | | | | | |
| P³ | −7.4 | −4.3 | −4.7 | | | | | | |
| P⁴ | 0.40 | −12 | −12 | | | | | | |
| P⁵ | −11 | −1.1 | −1.5 | | | | | | |
| P⁶ (Pro) | | −36 | | | | | | | |

Peptides:
1, H-Arg-Arg-Leu-Ile-Phe-NH₂ (SEQ. ID No: 2);
2, H-Arg-Asn-Leu-Phe-Gly-OH (SEQ. ID No: 9);
3, H-Arg-Arg-Leu-Phe-NH₂ (SEQ. ID No: 20).
*Water-bridged H-bond between the peptide Leu³ (CO) and Gln²⁵⁴ side chain (FIG. 3a) present only in p21^{WAF1} motif.

1. A method of preparing a crystal comprising CDK2/cyclin A and a ligand, L, said method comprising the steps of:
(i) co-crystallising CDK2/cyclin A and a first ligand, L′, to form a crystal;
(ii) removing at least a portion of said first ligand, L′ from said crystal;
(iii) contacting said crystal with a second ligand, L, to form a crystal comprising CDK2/cyclin A and ligand, L.

2. A method according to claim 1 wherein step (ii) comprises which comprises eluting the crystal obtained in step (i) with a solvent.

3. A method according to claim 1 wherein step (iii) comprises soaking the crystal obtained in step (ii) with a solution of ligand, L.

4. A method according to claim 1 wherein steps (ii) and (iii) are carried out sequentially.

5. A method according to claim 1 wherein steps (ii) and (iii) are carried out simultaneously.

6. A method according to claim 1 wherein step (ii) comprises eluting said first crystal with a solvent to remove substantially all of said first ligand, L′.

7. A method according to claim 1 wherein said first ligand, L′, and said second ligand, L, are different and are each capable of binding to the cyclin binding groove of CDK2/cyclin A.

8. A method according to claim 1 wherein said first ligand, L′, and said second ligand, L, are each cyclin binding groove inhibitors.

9. A method according to claim 8 wherein the cyclin binding groove is defined by the structural coordinates of the following amino acid residues of CDK2/cyclin A: Met²¹⁰, Ile²¹³, Leu²¹⁴, Trp²¹⁷, Leu²⁵³, Glu²²⁰, Val²²¹, Ile²⁸¹ and Gln²⁵⁴, or a homologue thereof.

10. A method according to claim 1 wherein said first ligand, L′, and said second ligand, L, are each peptides.

11. A method according to claim 10 wherein said first ligand, L′, and said second ligand, L, are each pentamers.

12. A method according to claim 1 wherein said first ligand, L′, and said second ligand, L, are selected from:
(SEQ ID NO. 1)
H-Ala-Ala-Abu-Arg-Ser-Leu-Ile-(p-F-Phe)-NH₂;
(SEQ ID NO. 2)
H-Arg-Arg-Leu-IIe-Phe-NH₂;
(SEQ ID NO. 3)
Ac-Arg-Arg-Leu-Asn-(m-Cl-Phe)-NH₂;
(SEQ ID NO. 4)
H-Arg-Arg-Leu-Asn-(p-F-Phe)-NH₂;
and
(SEQ ID NO. 5)
H-Cit-Cit-Leu-Ile-(p-F-Phe)-NH₂;
wherein L and L' are different.

13. A method according to claim 1 or 12 wherein said first ligand, L′, is selected from:

| | |
|---|---|
| H-Arg-Arg-Leu-Ile-Phe-NH₂; | (SEQ ID NO. 2) |
| and | |
| H-Arg-Arg-Leu-Asn-(p-F-Phe)-NH₂. | (SEQ ID NO. 4) |

14. A method according to claim 1 or 12 wherein said second ligand, L, is selected from:
(SEQ ID NO. 1)
H-Ala-Ala-Abu-Arg-Ser-Leu-Ile-(p-F-Phe)-NH₂;
(SEQ ID NO. 3)
Ac-Arg-Arg-Leu-Asn-(m-Cl-Phe)-NH₂;
and
(SEQ ID NO. 5)
H-Cit-Cit-Leu-Ile-(p-F-Phe)-NH₂.

15. A method according to claim 11 wherein residues 1, 3 and 5 of ligand L are capable of interacting with the cyclin binding groove of CDK2/cyclin A.

16. A method according to claim 11 wherein residue 1 of ligand L is capable of interacting with Glu²²⁰ of the cyclin binding groove of CDK2/cyclin A.

17. A method according to claim 16 wherein residue 1 is Arg.

18. A method according to claim 11 wherein residue 3 of ligand L is capable of interacting with Gln²⁵⁴, Trp²¹⁷ and Leu ²¹⁴ of the cyclin binding groove of CDK2/cyclin A.

19. A method according to claim 18 wherein residue 3 is Leu.

20. A method according to claim 1 wherein said crystal is of space group P2₁2₁ 2₁.

21. A method according to claim 12, wherein ligand L is H-Ala-Ala-Abu-Arg-Ser-Leu-Ile-(p-F-Phe)-NH₂ (SEQ ID NO. 1) and said crystal comprises a unit cell having unit dimensions a=74.5 Å, b=114.6 Å, c=157.0 Å.

22. A method according to claim 12, wherein ligand L is H-Arg-Arg-Leu-Ile-Phe-NH₂ (SEQ ID NO. 2) and said crystal comprises a unit cell having unit dimensions a=74.2 Å, b=113.9 Å, c=155.3 Å.

23. A method according to claim 12, wherein ligand L is Ac-Arg-Arg-Leu-Asn-(m-Cl-Phe)-NH₂ (SEQ ID NO. 3) and said crystal comprises a unit cell having unit dimensions a=74.5 Å, b=114.0 Å, c=156.2 Å.

24. A method according to claim 12, wherein ligand L is H-Arg-Arg-Leu-Asn-(p-F-Phe)-NH₂ (SEQ ID NO. 4) and said crystal comprises a unit cell having unit dimensions a=73.5 Å, b=113.0 Å, c=153.1 Å.

25. A method according to claim 12, wherein ligand L is H-Cit-Cit-Leu-Ile-(p-F-Phe)-NH₂ (SEQ ID NO. 5) and said crystal comprises a unit cell having unit dimensions a=74.5 Å, b=113.5 Å, c=154.5 Å.

26. A crystal obtainable by the method of claim 1.

27. A method for treating comprising administering a ligand that binds to the cyclin binding groove of CDK2/cyclin A, such that the cell proliferative disorder is treated.

28. A method for identifying ligands capable of binding to the cyclin binding groove of CDK2/cyclin A, comprising
(a) contacting a first crystal comprising CDK2/Cyclin and a first ligand L with a second ligand, L″
(b) detecting a second crystal comprising CDK/Cyclin A and the second ligand L;
wherein the formation of the second crystal is indicative that the second ligand L binds to the cyclin binding groove.

29. The method according to claim 28 which is a competitive binding assay.

30. A crystal comprising CDK2/cyclin A and a cyclin binding groove peptide selected from the following:
(SEQ ID NO. 1)
H-Ala-Ala-Abu-Arg-Ser-Leu-Ile-(p-F-Phe)-NH₂;
(SEQ ID NO. 2)
H-Arg-Arg-Leu-Ile-Phe-NH₂;
(SEQ ID NO. 3)
Ac-Arg-Arg-Leu-Asn-(m-Cl-Phe)-NH₂;
(SEQ ID NO. 4)
H-Arg-Arg-Leu-Asn-(p-F-Phe)-NH₂;
(SEQ ID NO. 5)
H-Cit-Cit-Leu-Ile-(p-F-Phe)-NH₂;
and
(SEQ ID NO. 6)
H-Arg-Arg-Leu-Ile-(p-F-Phe)-NH₂.

31. A crystal according to claim 30 wherein the crystal is of space group P2₁2₁2₁.

32. A crystal according to claim 30 wherein the cyclin binding groove is defined by the structural coordinates of the following amino acid residues of CDK2/cyclin A: Met²¹³, Ile²¹³, Leu²¹⁴, Trp²¹⁷, Leu²⁵³, Glu²²⁰, Val²²¹, Ile²⁸¹ and Gln²⁵⁴, or a homologue thereof.

33. A crystal according to claim 30 wherein residues 1, 3 and 5 of the cyclin binding groove peptide are capable of interacting with the cyclin binding groove of CDK2/cyclin A.

34. A crystal according to claim 30 wherein residue 1 of the cyclin binding groove peptide is capable of interacting with Glu²²⁰ of the cyclin binding groove of CDK2/cyclin A.

35. A crystal according to claim 30 wherein residue 3 of the cyclin binding groove peptide is capable of interacting with Gln²⁵⁴, Trp²¹⁷ and Leu²¹⁴ of the cyclin binding groove of CDK2/cyclin A.

36. A crystal according to claim 30, wherein ligand L is H-Ala-Ala-Abu-Arg-Ser-Leu-Ile-(p-F-Phe)-NH₂ (SEQ ID NO. 1) and said crystal comprises a unit cell having unit dimensions a=74.5 Å, b=114.6 Å, c=157.0 Å.

37. A crystal according to claim 30, wherein ligand L is H-Arg-Arg-Leu-Ile-Phe-NH₂ (SEQ ID NO. 2) and said crystal comprises a unit cell having unit dimensions a=74.2 Å, b=113.9 Å, c=155.3 Å.

38. A crystal according to claim 30, wherein ligand L is Ac-Arg-Arg-Leu-Asn-(m-Cl-Phe)-NH₂ (SEQ ID NO. 3) and said crystal comprises a unit cell having unit dimensions a=74.5 Å, b=114.0 Å, c=156.2 Å.

39. A crystal according to claim 30, wherein ligand L is H-Arg-Arg-Leu-Asn-(p-F-Phe)-NH₂ (SEQ ID NO. 4) and said crystal comprises a unit cell having unit dimensions a=73.5 Å, b=113.0 Å, c=153.1 Å.

40. A crystal according to claim 30, wherein ligand L is H-Cit-Cit-Leu-Ile-(p-F-Phe)-NH₂ (SEQ ID NO. 5) and said crystal comprises a unit cell having unit dimensions a=74.5 Å, b=113.5 Å, c=154.5 Å.

41. An assay for screening one or more ligands capable of binding to the cyclin binding groove of CDK2/cyclin A, said assay comprising contacting a crystal according to claim 30, or a crystal obtainable by the method according to claim 1, with a candidate ligand L″, and detecting any change in the interaction between CDK2/cyclin A and ligand L.

42. An assay according to claim 41 which is used to screen for a ligand useful as an inhibitor of CDK2/Cyclin A.

43. An assay according to claim 41 which is used to screen for a ligand useful in the prevention and/or treatment of a proliferative disorder.

44. An assay according to claim 41 which is used to screen for a ligand useful in the prevention and/or treatment of cancer, leukemia, a viral disorder, stroke, glomerulonephritis, alopecia, diabetes, a CNS disorder, rheumatoid arthritis, psoriasis or chronic obstructive pulmonary disorder.

45. A process comprising the steps of:
(a) performing the assay according to claim 41;
(b) identifying one or more ligands capable of binding to the cyclin binding groove; and
(c) preparing a quantity of said one or more-ligands.

46. A process comprising the steps of:
(a) performing the assay according to claim 41;
(b) identifying one or more ligands capable of binding to the cyclin binding groove; and
(c) preparing a pharmaceutical composition comprising said one or more ligands.

47. A process comprising the steps of:
(a) performing the assay according to claim 41;
(b) identifying one or more ligands capable of binding to the cyclin binding groove;
(c) modifying said one or more ligands capable of binding to the cyclin binding groove;
(d) performing said assay according to claim 41;
(e) optionally preparing a pharmaceutical composition comprising said one or more ligands.

48. A ligand identified by the method of claim 41.

49. A pharmaceutical composition comprising the ligand of claim 48.

50. A method for treating a cell proliferative disorder comprising administering the pharmaceutical composition of claim 49.

51. The method of claim 50, wherein the cell proliferative disorder is cancer, leukemia, a viral disorder, stroke, glomerulonephritis, alopecia, diabetes, a CNS disorder, rheumatoid arthritis, psoriasis or chronic obstructive pulmonary disorder.


SEQUENCE LISTING (31 sequences)

SEQ ID NO: 1 - protein, 8 aa, Artificial Sequence
  Description of Artificial Sequence: Synthetic Peptide
  MOD_RES (3): Abu
  MOD_RES (8): p-F-Phe
AAXRSLIX

SEQ ID NO: 2 - protein, 5 aa, Artificial Sequence
  Description of Artificial Sequence: Synthetic Peptide
RRLIF

SEQ ID NO: 3 - protein, 5 aa, Artificial Sequence
  Description of Artificial Sequence: Synthetic Peptide
  MOD_RES (5): m-CL-Phe
RRLNX

SEQ ID NO: 4 - protein, 5 aa, Artificial Sequence
  Description of Artificial Sequence: Synthetic Peptide
  MOD_RES (5): p-F-Phe
RRLNX

SEQ ID NO: 5 - protein, 5 aa, Artificial Sequence
  Description of Artificial Sequence: Synthetic Peptide
  MOD_RES (1): Cit
  MOD_RES (2): Cit
  MOD_RES (5): p-F-Phe
XXLIX

SEQ ID NO: 6 - protein, 5 aa, Artificial Sequence
  Description of Artificial Sequence: Synthetic Peptide
  MOD_RES (5): p-F-Phe
RRLIX

SEQ ID NO: 7 - protein, 4 aa, Artificial Sequence
  Description of Artificial Sequence: Synthetic Peptide
  MOD_RES (1): basic residue
  MOD_RES (3): basic residue
XRXL

SEQ ID NO: 8 - protein, 6 aa, Artificial Sequence
  Description of Artificial Sequence: Synthetic Peptide
  MOD_RES (1): basic residue
  MOD_RES (3): basic residue
  MOD_RES (5): hydrophobic residue
  MOD_RES (6): hydrophobic residue
XRXLXX

SEQ ID NO: 9 - protein, 5 aa, Artificial Sequence
  Description of Artificial Sequence: Synthetic Peptide
RNLFG

SEQ ID NO: 10 - protein, 11 aa, Artificial Sequence
  Description of Artificial Sequence: Synthetic Peptide
RRLFGEDPPKE

SEQ ID NO: 11 - protein, 11 aa, Artificial Sequence
  Description of Artificial Sequence: Synthetic Peptide
STSRHKKLMFK

SEQ ID NO: 12 - protein, 11 aa, Artificial Sequence
  Description of Artificial Sequence: Synthetic Peptide
PPKPLKKLRFD

SEQ ID NO: 13 - protein, 11 aa, Artificial Sequence
  Description of Artificial Sequence: Synthetic Peptide
KPSACRNLFGP

SEQ ID NO: 14 - protein, 9 aa, Artificial Sequence
  Description of Artificial Sequence: Synthetic Peptide
PVKRRLDLE

SEQ ID NO: 15 - protein, 11 aa, Artificial Sequence
  Description of Artificial Sequence: Synthetic Peptide
AGSAKRRLFGE

SEQ ID NO: 16 - protein, 5 aa, Artificial Sequence
  Description of Artificial Sequence: Synthetic Peptide
RRLIF

SEQ ID NO: 17 - protein, 8 aa, Artificial Sequence
  Description of Artificial Sequence: Synthetic Peptide
HAKRRLIF

SEQ ID NO: 18 - protein, 5 aa, Artificial Sequence
  Description of Artificial Sequence: Synthetic Peptide
  MOD_RES (2): basic residue
  MOD_RES (4): hydrophobic residue
  MOD_RES (5): hydrophobic residue
RXLXX

SEQ ID NO: 19 - skipped

SEQ ID NO: 20 - protein, 4 aa, Artificial Sequence
  Description of Artificial Sequence: Synthetic Peptide
RRLF

SEQ ID NO: 21 - protein, 5 aa, Artificial Sequence
  Description of Artificial Sequence: Synthetic Peptide
  MOD_RES (5): p-F-Phe
RRLNX

SEQ ID NO: 22 - protein, 5 aa, Artificial Sequence
  Description of Artificial Sequence: Synthetic Peptide
  MOD_RES (2): Cit
  MOD_RES (5): p-F-Phe
RXLIX

SEQ ID NO: 23 - protein, 5 aa, Artificial Sequence
  Description of Artificial Sequence: Synthetic Peptide
  MOD_RES (5): p-F-Phe
RRLAX

SEQ ID NO: 24 - protein, 5 aa, Artificial Sequence
  Description of Artificial Sequence: Synthetic Peptide
  MOD_RES (2): Cit
  MOD_RES (5): p-F-Phe
RXLAX

SEQ ID NO: 25 - protein, 5 aa, Artificial Sequence
  Description of Artificial Sequence: Synthetic Peptide
  MOD_RES (5): p-F-Phe
RQLIX

SEQ ID NO: 26 - protein, 8 aa, Artificial Sequence
  Description of Artificial Sequence: Synthetic Peptide
HAKRRAIF

SEQ ID NO: 27 - protein, 5 aa, Artificial Sequence
  Description of Artificial Sequence: Synthetic Peptide
  MOD_RES (1): Cit
  MOD_RES (2): Cit
  MOD_RES (5): p-F-Phe
XXLAX

SEQ ID NO: 28 - protein, 5 aa, Artificial Sequence
  Description of Artificial Sequence: Synthetic Peptide
  MOD_RES (1): Cit
  MOD_RES (2): Cit
  MOD_RES (5): p-F-Phe
XXLNX

SEQ ID NO: 29 - protein, 4 aa, Artificial Sequence
  Description of Artificial Sequence: Synthetic Peptide
  MOD_RES (4): p-F-Phe
RRLX

SEQ ID NO: 30 - protein, 5 aa, Artificial Sequence
  Description of Artificial Sequence: Synthetic Peptide
RRLNF

SEQ ID NO: 31 - protein, 5 aa, Artificial Sequence
  Description of Artificial Sequence: Synthetic Peptide
  MOD_RES (5): p-Cl-Phe
RRLNX